(19) United States
(12) Patent Application Publication
(10) Pub. No.: US 2026/0257992 A1
(43) Pub. Date: Sep. 3, 2026

(54) AGONISTS OF SMALL HETERODIMER PARTNER FOR CANCER THERAPY

(71) Applicant: THE BOARD OF TRUSTEES OF THE UNIVERSITY OF ILLINOIS, Urbana, IL (US)

(72) Inventors: Erik Russell NELSON, Champaign, IL (US); Paul J. HERGENROTHER, Champaign, IL (US); Hashni Epa Vidana GAMAGE, Urbana, IL (US); Samuel ALBRIGHT, Urbana, IL (US); Rachel FARMER, Evansville, IN (US); Sayyed Hamed SHAHOEI, West Hartford, CT (US)

(73) Assignee: THE BOARD OF TRUSTEES OF THE UNIVERSITY OF ILLINOIS, Urbana, IL (US)

(21) Appl. No.: 19/474,194

(22) PCT Filed: Apr. 11, 2024
(86) PCT No.: PCT/US2024/024094

§ 371 Date: Oct. 10, 2025

Related U.S. Application Data
(60) Provisional application No. 63/458,834, filed on Apr. 12, 2023.
(60) Provisional application No. 63/519,473, filed on Aug. 14, 2023.

(51) Int. Cl. C07C 311/29; A61K 31/24; A61K 31/403; A61P 35/00; C07D 209/92

(52) U.S. Cl. CPC C07C 311/29; A61K 31/24; A61K 31/403; A61P 35/00; C07D 209/92

(57) ABSTRACT

Novel compounds that are improved analogs and mimetics of DSHN and methods to treat or inhibit cancer using the compounds. The methods can require lower doses than for DSHN and the compounds can have a larger biological maximum than DSHN. Nuclear receptor NR0B2 is activated by the disclosed compounds to reduce immune-suppressive T_{regs}, thereby attenuating tumor growth and metastasis. The therapy disclosed herein results in an inhibition of T_{reg} expansion and an expansion of cytotoxic T cells. This therapy can significantly reduce tumor growth with lower doses compared to other available treatments.

### RELATED APPLICATIONS

This application claims priority under 35 U.S.C. § 119(e) to U.S. Provisional Patent Application Nos. 63/519,473, filed Aug. 14, 2023, and 63/458,834, filed Apr. 12, 2023, which applications are incorporated herein by reference.

### GOVERNMENT SUPPORT

This invention was made with government support under W81XWH2110177 awarded by the Department of Defense. The government has certain rights in the invention.

### BACKGROUND OF THE INVENTION

In the last decade, immune therapy has revolutionized cancer treatment. Chimeric antigen receptor (CAR) T cells are remarkably effective but are limited to tumors with known and unique antigens, or at least antigens on normal cells that are dispensable for life (i.e., CD19 on B cells). In theory, immune checkpoint blockade (ICB) promised to apply to all solid tumors. However, in practice, it is only effective in certain tumor types or subtypes. Breast cancer is an example of this, where ICB has proven to be largely unsuccessful, and is currently only approved for those diagnosed with triple negative breast cancer (TNBC) whose tumors also stain positive for Programmed death-ligand 1 (PD-L1). Even amongst these patients receiving anti PD-1 (αPD1) in combination with nab-paclitaxel, only 20-30% show response with the majority being refractory. Genentech recently announced that it will voluntarily withdraw atezolizumab (αPD-L1) in combination with nab-paclitaxel for consideration of accelerated approval for metastatic PD-L1-positive TNBC (Clinical Cancer Research, August 2021). Unfortunately, since TNBC lacks endocrine targets such as the estrogen receptor (ERα) or HER2, no targeted therapies exist other than for BRCA1/2 mutant tumors, resulting in a high recurrence and mortality rate.

Therefore, there is urgent need to better understand alternate immune-suppressive networks in solid tumors such as TNBC and develop therapies that combine ICB with pro-immune drugs that work in a checkpoint-independent manner.

### SUMMARY

We have developed a novel immune therapy that targets a nuclear receptor to reduce the growth of immunosuppressive T-cells in breast cancer. The nuclear receptor, myeloid cell NR0B2 (also known as small heterodimer partner or SHP), directs away from regulatory T cells and promotes anti-tumor efficacy in breast cancer. The developed therapy has shown excellent potential for future cancer treatments. It has significantly reduced tumor growth in mouse models with lower doses compared to other available treatments.

This disclosure provides new compounds that are improved analogs and mimetics of DSHN. The disclosure also provides methods to treat or inhibit cancer using these compounds. The methods can require lower doses than for DSHN and the compounds can have a larger biological maximum than DSHN. The treatment can result in inhibition of T_{reg} expansion and expansion of cytotoxic T cells. The therapy has significantly reduced tumor growth in mouse models with lower doses compared to other available treatments.

Accordingly, this Disclosure Provides a Compound of Formula I:

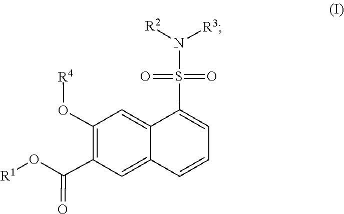

wherein
R¹ is (C₁-C₈)alkyl or H;
R² is (C₁-C₈)alkyl or H;
R³ is (C₁-C₈)alkyl or H; or
   R² and R³ taken together with the nitrogen atom to which they are attached form a 3- to 9-membered ring heterocycle; and
R⁴ is (C₁-C₈)alkyl or H;
   wherein the naphthalene moiety of Formula I and each (C₁-C₈)alkyl are optionally substituted with one or more substituents;
or a pharmaceutically acceptable salt thereof.

Such compounds can regulate T_{reg} cell expansion. Therefore, this disclosure also provides a method of treating cancer comprising administering to a patient in need thereof an effective amount of a compound of Formulas I-III.

The technology disclosed herein provides novel compounds of Formulas I-III, intermediates for the synthesis of compounds of Formulas I-III, as well as methods of preparing compounds of Formulas I-III. The technology also provides compounds of Formulas I-III that are useful as intermediates for the synthesis of other useful compounds. The technology provides for the use of compounds of Formulas I-III for the manufacture of medicaments useful for the treatment of cancer or bacterial infections in a mammal, such as a human.

The technology also provides for the use of the compounds and compositions described herein for use in medical therapy. The therapy can be treating cancer, for example, breast cancer, lung cancer, pancreatic cancer, prostate cancer, colon cancer, or other cancers recited herein. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention described herein.

FIG. 1A-1G. NR0B2 mRNA expression within breast and lung tumors is associated with increased recurrence free survival time. (A) All breast cancer subtypes are considered. (B) Lung cancer using the Kaplan Meier plotter, autocutoff. (C) NR0B2 mRNA expression within tumors from patients treated with immune checkpoint blockers (ICB) is associated with an increased progression free survival time. The Kaplan-Meier plotter was used to probe associations between NR0B2 expression in both genders and all cancers within the database. (D) Loss of NR0B2 in murine bone marrow derived macrophages (BMDMs) results in increased T_{reg} expansion in T cells activated by αCD3, IL-2 and TGFβ (N=3/group, t test). (E) CD4+ T cells expanded in co-culture with BMDMs lacking NR0B2 (NR0B2^{fl/fl}; LysMcre) suppressed CD8+ T cell expansion compared to those co-cultured with control (NR0B2^{+/+}; LysMcre) macrophages. Expanded CD4+ T cells were cocultured at different ratios with splenic CD8+ T cells that had been activated with CD3/28 Dynabeads and subsequent proliferation of CD8+ cells was assessed. (F) DCs derived from PBMCs of healthy human volunteers resulted in decreased T_{reg} expansion in T cells when treated with DSHN, a NR0B2 agonist (N=3/group). (G) FoxP3 expression is lower in human tumors with high NR0B2 expression (upper quartile compared to lower quartile depicted here, N=477-478/group, t test). Data obtained from METABRIC.

FIG. 2A-2E. Loss of NR0B2 results in increased T_{regs} and tumor growth in murine models. (A) Germline deletion of NR0B2 promotes growth of spontaneous mammary tumors in MMTV-PyMT mice. Time from birth to developing a tumor burden of >2000 mm³ is depicted. NR0B2^{-/-}; PyMT⁺ and NR0B2^{+/+}; PyMT⁺ littermates were monitored frequently after birth for development of mammary masses and subsequent tumor growth was assessed by caliper measurements (N=12 per group, Log-rank test). (B) MMTV-PyMT tumors from NR0B2^{-/-} mice had increased T_{regs} (CD4⁺; CD25⁺; FoxP3⁺) and decreased T_{eff} (CD4⁺; IFN-γ*) cells compared to wildtype controls (N=3-6/group, t test). (C) Orthotopic, syngeneic E0771 mammary tumors grew at a faster rate in mice with NR0B2 selectively knocked out in myeloid cells compared to control mice (NR0B2^{fl/fl}; LysMCre⁺ vs. NR0B2^{+/+}; LysMCre⁺). Tumor growth curve shown above the final tumor weight at necropsy (N=10-15/group, 2-way ANOVA followed by Šidák's multiple comparison, or t test). (D) In a separate experiment, tumors isolated from NR0B2^{fl/fl}; LysMCre⁺ mice had significantly more T_{reg} cells compared to NR0B2^{+/+}; LysMCre⁺ control mice (N=10-11/group, t test). (E) Murine melanoma B16-F0 tumors grew at a faster rate in NR0B2^{fl/fl}; LysMCre⁺ compared to NR0B2^{+/+}; LysMCre⁺ control mice. Tumor growth curve shown above the final tumor weight at necropsy (N=12-16/group, 2-way ANOVA followed by Šidák's multiple comparison, or t test).

FIG. 3A-3F. DSHN, a tool small molecule agonist of NR0B2 reduces macrophage/dendritic cell induced T_{reg} expansion and tumor growth in murine models. (A) Co-culture of DSHN pre-treated BMDMs results in decreased T_{reg} expansion in a dose dependent manner. (B) Dendritic cells (left-CD11B⁺; CD11C⁺) and CD4⁺ T cells (right) were isolated from MMTV-PyMT tumors by FACS, and treated with vehicle or DSHN for 3d. The resulting frequency of T_{reg} (CD4⁺; CD25⁺; FoxP3⁺) was quantified by flow cytometry (right graph), (N=3-5/group, t test). (C) T cells expanded in the presence of DSHN treated BMDMs were less suppressive than those expanded in the presence of vehicle treated BMDMs, in terms of a subsequent co-culture of naïve T cells. The percentage of divided CD8+ T cells in the second round was quantified. (D) DSHN increases efficacy of αPDL1 therapy on orthotopic E0771 tumors. An optimal number of E0771 cells were grafted into FoxP3-EGFP mice and allowed to grow until tumors reached 100 mm³. Mice were euthanized on day 19 (waterfall plot, Fisher's exact test). (E) DSHN increases efficacy of αPDL1 therapy on orthotopic 4T1 tumors. 4T1 cells were orthotopically grafted into mice and allowed to establish for 5d prior to treatment start. Mice were euthanized on day 16 (waterfall plot, Fisher's exact test). (F) DSHN reduces metastatic outgrowth of 4T1 tumors and enhances efficacy of αPDL1 compared to placebo (p) (2-way ANOVA followed by Šidák's multiple comparison test for day 16). 4T1 cells were grafted intravenously, and lung metastatic lesions allowed to establish for 3d before treatment commenced. At the end of the study, lungs were removed and imaged ex vivo, quantified data indicated (N=10, 1-way ANOVA followed by Newman-Keuls multiple comparison test). Representative luciferase images shown to the right. * or different letters denote P<0.05 using indicated statistical test.

FIG. 4A-4L. NR0B2 regulates many aspects of the inflammasome resulting in altered T_{reg} expansion. (A) Ca²⁺ concentration decreases in ionomycin-treated BMDMs overexpressing NR0B2 (N=3/group, t test), and (B) increased when NR0B2 is knocked out. (C) Reactive oxygen species (ROS) is increased in BMDMs where NR0B2 is knocked out (N=3/group, t test). (D) S100A8 and S100A9 transcript levels are increased in CD11B+ cells isolated from tumors where NR0B2 was knocked out of the myeloid cell lineage (N=4/group, t test). (E) Lysosome stability is decreased in BMDMs lacking NR0B2. Data quantified from mean fluorescence intensity (MFI, N=4/group). (F) Left panel: secreted cathepsin from control BMDMs or those overexpressing NR0B2 (+). Right panel: secreted cathepsin from control BMDMs (+/+) or those lacking NR0B2 (fl/fl). (N=4/group, t test) as indicated. (G) Intracellular caspase 1 activity in BMDMs transfected with control or NR0B2 expression plasmid (N=5/group, t test). (H) Intracellular and secreted caspase 1 activity in CD11B+ cells isolated from E0771 tumors. (I & J) IL-1β secreted protein from BMDMs (N=4/group, t test). (K) siRNA against caspase 1 or IL-1β attenuates induction of T_{regs} in co-culture with myeloid cells from NR0B1^{fl/fl}; LysMcre⁺ mice (N=5/group) (L) Immune neutralization with αIL-1β attenuates induction of T_{regs} in co-culture with myeloid cells from NR0B1^{fl/fl}; LysMcre⁺ mice.

FIG. 5A-5D. DSHN-OMe, an NR0B2 small molecule ligand with increased potency in terms of reducing T_{reg} expansion and strong efficacy against metastatic mammary tumors. (A) DSHN-OMe effectively reduces induction of LXR target gene ABCA1 by GW3965. Different letters denote statistical difference between GW3965 treated groups (N=3-4/group, 1-way ANOVA followed by Šidák test). (B) Dose response assay in BMDMs pre-treated with increasing doses of DSHN-OMe and then treated with 1 μM GW3965, using ABCA1 mRNA as an endpoint. Data was normalized to 0.01 μM DSHN-OMe (0%) and GW3965 alone (100%). DSHN (100 μM) is indicated for comparison (bar at end of graph). Data was fit to a 4 parameter variable slope model, and IC₅₀ estimated at 3.8 μM. (C) Thermal shift assay using a recombinant protein presence of a fluorescent dye. Plot of raw data is above plot of first derivative. (D) DSHN-OMe is ineffective at regulating ABCA1 when NR0B2 is knocked down with siRNA in BMDMs.

FIG. 6A-6F. DSHN-OMe has significantly improved cellular uptake and works on myeloid cells to reduce T_{reg} expansion. (A) DSHN-OMe has minimal effects on BMDM viability (N=4-6/group). (B) DSHN-OMe treated BMDMs reduce T_{reg} expansion in a dose related manner, with an increased biological maximum compared to DSHN (data normalized to mean vehicle, which was set at 100%. Non-linear three parameter regression shown, N=4/point). (C) DSHN-OMe treated splenic derived CD11C+ cells result in decreased T_{reg} expansion compared to twice the dose of DSHN. 50 μM DSHN-OMe treated CD11C+ cells primed with OVA results in decreased T_{reg} expansion of OTI T cells compared to vehicle (DMSO) or 100 μM DSHN. Different letters denote P<0.05. (D) T cells expanded in the presence of DSHN- or DSHN-OMe-treated BMDMs were less suppressive than those expanded in the presence of vehicle treated BMDMs, in terms of a subsequent co-culture of naïve T cells. The percentage of divided CD8+ T cells in the second round was quantified. (N=3-5/group, different letters indicating statistical significance, 1-way ANOVA followed by Tukey). (E) BMDMs pretreated with DSHN-OMe for 24 h prior to washout and subsequent co-culture results in decreased T_{reg} expansion, while DSHN does not. Vehicle (DMSO) treated BMDMs are indicated on the right for comparison. (F) Cellular uptake of DSHN-OMe greatly exceeds that of DSHN though time. RAW264.7 cells were treated with 50 M of vehicle (DMSO) or indicated compound for 4 h, 8 h or 24 h. Compound was washed off, cells were lysed and resulting DSHN and DSHN-OMe contents were assessed by LC-MS/MS. Note different scales on y-axes. For DSHN treated cells, no DSHN-OMe was detected (not shown). For DSHN-OMe treated cells, some DSHN was detected. ND signifies when compounds were not detected. (N=3/group. 4 h timepoint was run in an experiment independent of the 8 and 24 hr timepoints).

FIG. 7A-7I. DSHN-OMe reduces tumor and metastatic growth in murine models, in a myeloid cell-NR0B2 dependent manner. (A) E0771 cells were grafted into control mice (NR0B2^{+/+}; LysMCre⁺) or mice where NR0B2 was knocked out in myeloid immune cells (NR0B2^{fl/fl}; LysMCre⁺). 8 days post-graft, daily treatment with placebo or DSHN-OMe was initiated and subsequent tumor growth followed through time. Different letters denote statistical difference on the final measurement (day 21, 2-Way ANOVA followed by Šidák's post-hoc, N=5-8). (B) Waterfall plot illustration of data in (A) showing tumor volume at final day of measurement. Asterisk indicates significant difference (Chi-squared test, P<0.05). (C) Lewis Lung cells were grafted into control mice (NR0B2^{+/+}; LysMCre⁺) or mice where NR0B2 was knocked out in myeloid immune cells (NR0B2^{fl/fl}; LysMCre⁺). 10 days post-graft, daily treatment with placebo or DSHN-OMe was initiated and subsequent tumor growth followed through time. Different letters denote statistical difference on the final measurement (day 21, 2-Way ANOVA followed by Šidák's post-hoc, N=8-13). (D) Waterfall plot illustration of data in (C) showing tumor volume at final day of measurement. Asterisk indicates significant difference (Chi-squared test, P<0.05). (E) DSHN-OMe is an effective treatment against metastatic 4T1 mammary cancer growth. 4T1 cells were grafted intravenously, and lung metastatic lesions allowed to establish for 3d before treatment commenced. Metastatic burden through time is presented above the metastatic burden on the final day of imaging in the middle panel (N=12-17/group, mixed-effects model followed by Tukey's multiple comparison test). Representative luciferase images in the bottom panel. (F) DSHN-OMe is an effective treatment against metastatic EMT6 mammary cancer growth. EMT6 cells were grafted intravenously, and lung metastatic lesions allowed to establish for 3d. DSHN-OMe was administered on days 4, 5, 8, 9 and 10 post-graft. 11d post-graft lungs were imaged ex vivo for luciferase (N=15/group). (G) EMT6 metastatic lungs were assessed for Ki67 mRNA and (H) FOXP3 mRNA. (I) EMT6 metastatic lungs were assessed for CD8+ cytotoxic T cells (CD8+). Asterisks indicate statistically significant differences.

FIG. 8A-8D. DSHN-OMe attenuates induction of inflammasome associated genes when BMDMs are treated with both LPS and nigericin, not LPS alone. * indicates P<0.05 when comparing to DMSO.

### DETAILED DESCRIPTION

Immune checkpoint blockade (ICB) has revolutionized cancer therapy but has had limited utility in several solid tumors such as breast cancer, a major cause of cancer-related mortality in women. Therefore, there is considerable interest in alternate strategies to promote an anti-cancer immune response. We demonstrated that NR0B2, a protein involved in cholesterol homeostasis, functions within myeloid immune cells to modulate the NLRP3 inflammasome and reduce the expansion of immune-suppressive regulatory T cells (T_{reg}). Loss of NR0B2 increased mammary tumor growth and metastasis. Small molecule agonists, including one developed here, reduced T_{reg} expansion, reduced metastatic growth and improved the efficacy of ICB. This work identifies NR0B2 as a target to re-educate myeloid immune cells providing proof-of-principle that this cholesterol-homeostasis axis may have utility in enhancing ICB.

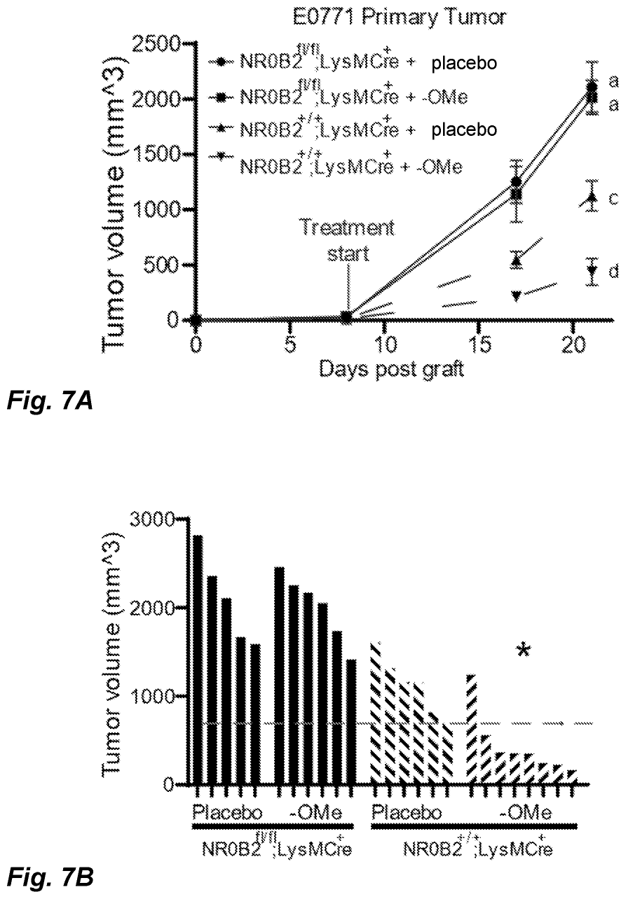

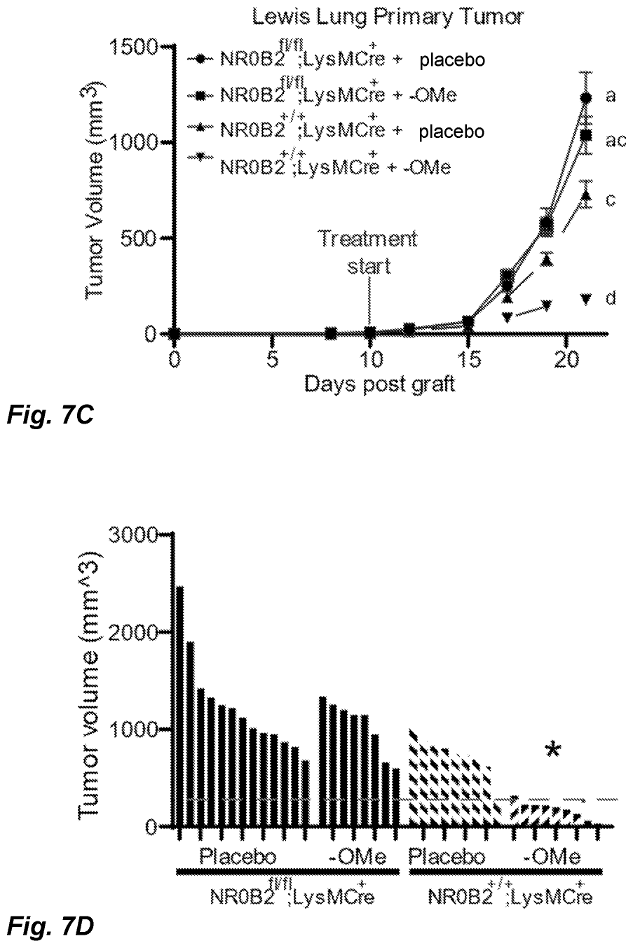

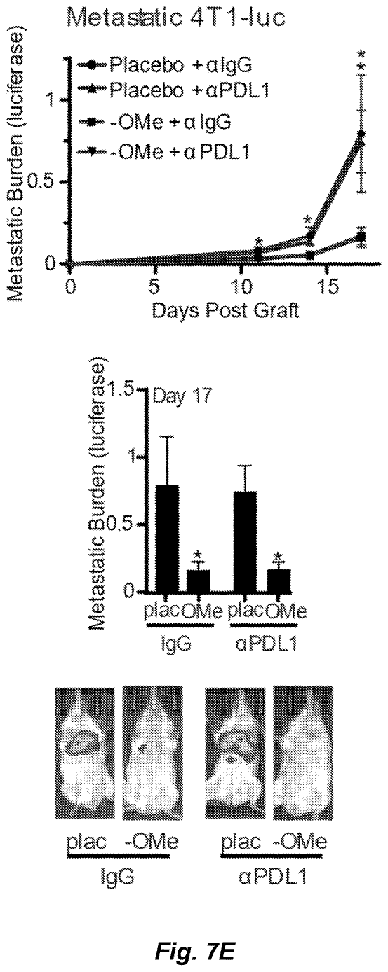

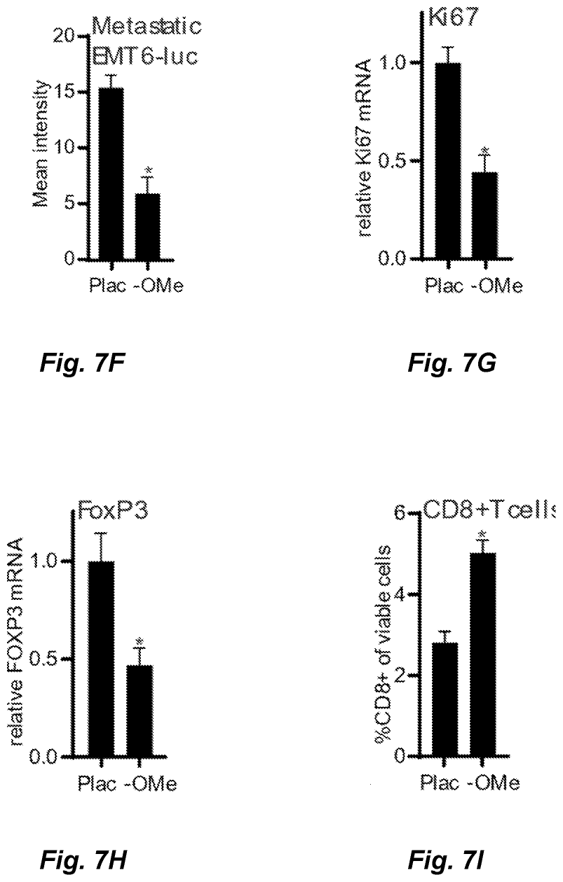

The compound DSHN-OMe was tested in two experimental models: 1) Primary E0771 tumors that are a murine mammary cancer syngeneic graft. Cells were grafted orthotopically into the mammary fat pad. Daily treatment started after palpable tumors formed on Day 8 (FIG. 7A). 2) Primary Lewis lung tumors that are a murine lung cancer syngeneic graft. Cells were grafted into the flank of male mice. Daily treatment started on Day 10 when tumors had started to become palpable (FIG. 7C).

For both models, we included control mice (NR0B2\hat{ }+/+; LysMcre⁺ (i.e., wildtype) to mice lacking NR0B2 expression in the myeloid immune cells (NR0B2\hat{ }fl/fl; LysMcre+). Tumors in the knockout mice were not responsive to DSHN-OMe. This indicated that DSHN-OMe is active ‘on-target’ through NR0B2 in the myeloid immune cells.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of +5%, +10%, +20%, or +25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, the patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl”, “heterocyclyl”, or “heterocycle” as referred to herein can be a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3- to 10-membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazepane, 1,4-diazepane, 1,4-oxazepane, and 1,4-oxathiepine. The group may be a terminal group or a bridging group.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents of indicated groups can be bonded to a substituted carbon atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety (e.g., (C₁-C₆)alkyl), and wherein the carbon-based moiety can itself be further substituted. When a substituent is monovalent, such as, for example, F or Cl, it is bonded to the atom it is substituting by a single bond. When a substituent is divalent, such as O, it is bonded to the atom it is substituting by a double bond; for example, a carbon atom substituted with O forms a carbonyl group, C═O.

The term “IC₅₀” is generally defined as the concentration required to inhibit a specific biological or biochemical function by half, or to kill 50% of the cells in a designated time period, typically 24 hours.

The term “microenvironment” as referred to herein is the environment within the immediate vicinity of cancer cell or the tumor, but not necessarily inside of the cancer cells.

The compound “DSHN” refers to the NR0B2 agonist having the structure:

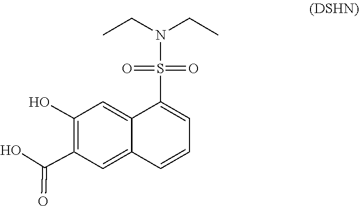 [SMILES: CCN(CC)S(=O)(=O)c1cccc2cc(C(=O)O)c(O)cc12]

### EMBODIMENTS OF THE TECHNOLOGY

### This Disclosure Provides a Compound of Formula I:

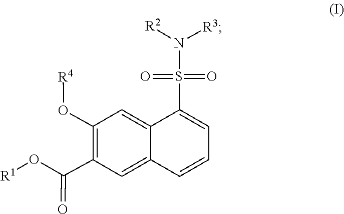

wherein
R¹ is (C₁-C₈)alkyl or H;
R² is (C₁-C₈)alkyl or H;
R³ is (C₁-C₈)alkyl or H; or
   R² and R³ taken together with the nitrogen atom to which they are attached form a 3-, 4-, 5-, 6-, 7-, 8-, or 9-membered ring heterocycle, optionally when R¹ is (C₁-C₈)alkyl; and
R⁴ is (C₁-C₈)alkyl or H;
   wherein the naphthalene moiety of Formula I and each (C₁-C₈)alkyl are optionally substituted with one or more substituents;
   or a pharmaceutically acceptable salt thereof.

In some embodiments, R² and R³ taken together with the nitrogen atom to which they are attached form a 3-, 4-, 5-, 6-, 7-, 8-, or 9-membered ring heterocycle provided that R¹ is (C₁-C₅)alkyl. In some embodiments, R¹ is (C₁-C₈)alkyl provided that R⁴ is H. In some embodiments, R¹ is (C₁-C₈)alkyl provided that R² or R³ is an alkyl and R⁴ is H. In some embodiments, R¹ is (C₁-C₈)alkyl when R² and R³ are both ethyl and R⁴ is H. In some embodiments, R¹ is (C₁-C₈)alkyl provided that R⁴ is H and R² and R³ taken together with the nitrogen atom to which they are attached form a 6- or 7-membered ring heterocycle. In other embodiments, R¹, R², R³, and R⁴ are each not H. In some embodiments, R¹ is (C₁-C₃)alkyl. In some embodiments, R² is (C₁-C₃)alkyl and R³ is (C₁-C₃)alkyl. In some embodiments, R¹ is methyl or ethyl. In some embodiments, R² and R³ are each ethyl, methyl, or propyl. In some embodiments, R¹ is methyl and R² and R³ are each ethyl or methyl. In some embodiments, R² and R³ taken together with the nitrogen atom to which they are attached form a piperidine, piperazine, pyrrolidine, morpholine, azepane, or azocane.

### In Some Embodiments, the Compound of Formula I is:

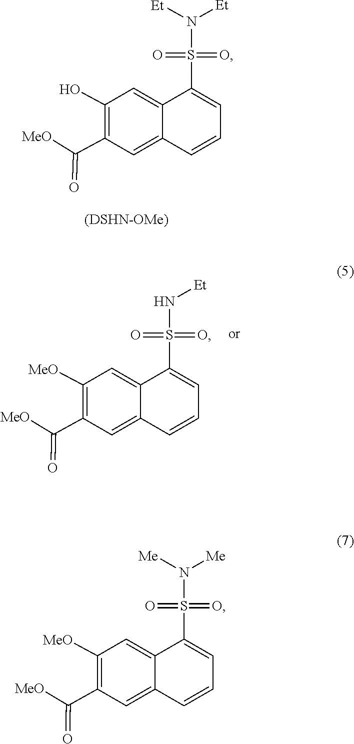 [SMILES: CCN(CC)S(=O)(=O)c1cccc2cc(C(=O)OC)c(O)cc12 ; "(DSHN-OMe)" ; CCNS(=O)(=O)c1cccc2cc(C(=O)OC)c(OC)cc12 ; "or" ; COC(=O)c1cc2cccc(S(=O)(=O)N(C)C)c2cc1OC]

or a pharmaceutically acceptable salt thereof.

### This Disclosure Also Provides a Compound of Formula II:

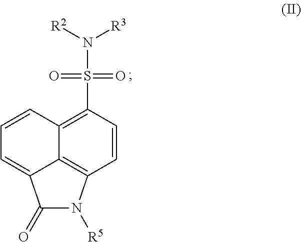

wherein
R² is (C₁-C₈)alkyl or H;
R³ is (C₁-C₈)alkyl or H; or
R² and R³ taken together with the nitrogen atom to which they are attached optionally form a 3- to 9-membered ring heterocycle; and
R⁵ is H or (C₁-C₈)alkyl;
   wherein the naphthalene moiety of Formula II and each (C₁-C₈)alkyl are optionally substituted with one or more substituents;
   or a pharmaceutically acceptable salt thereof.
   Additionally, this Disclosure Provides a Compound of Formula III:

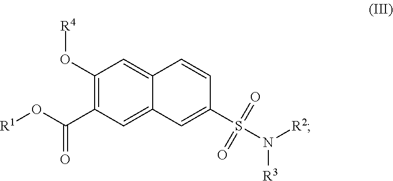

wherein
R¹ is (C₁-C₈)alkyl or H;
R² is (C₁-C₈)alkyl or H;
R³ is (C₁-C₈)alkyl or H; or
   R² and R³ taken together with the nitrogen atom to which they are attached form a 3-, 4-, 5-, 6-, 7-, 8-, or 9-membered ring heterocycle; and
R⁴ is (C₁-C₈)alkyl or H;
   wherein the naphthalene moiety of Formula I and each (C₁-C₈)alkyl are optionally substituted with one or more substituents;
   or a pharmaceutically acceptable salt thereof.

### In Some Embodiments, the Compound of Formula II is:

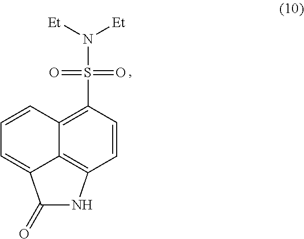 [SMILES: CCN(CC)S(=O)(=O)c1ccc2c3c(cccc13)C(=O)N2]

or a pharmaceutically acceptable salt thereof.

### In Some Embodiments, the Compound of Formula III is:

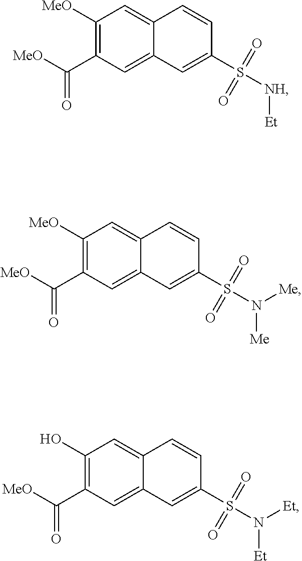 [SMILES: CCNS(=O)(=O)c1ccc2cc(OC)c(C(=O)OC)cc2c1 ; COC(=O)c1cc2cc(S(=O)(=O)N(C)C)ccc2cc1OC ; CCN(CC)S(=O)(=O)c1ccc2cc(O)c(C(=O)OC)cc2c1]

or a pharmaceutically acceptable salt thereof.

Furthermore, this disclosure provides a pharmaceutical composition comprising a compound of Formulas I-III and a pharmaceutically acceptable diluent or carrier. Compounds or compositions of Formula I-III can be used as anticancer agents for in-vivo or in-vitro methods of cancer treatment and diagnostic or screening assays.

This disclosure also provides a method of treating cancer comprising administering to a subject in need thereof a therapeutically effective amount of a compound of Formulas I-III, wherein the cancer is thereby treated. Compounds of Formula I-III act as anti-cancer agents.

Administering a compound of Formulas I-III results in inhibition of T_{reg} expansion. In some embodiments, the administering results in depletion of regulatory T cells. In some embodiments, the administering results in depletion of suppressor T cells. In some embodiments, the administering results in depletion of immune suppressive T cells. In some embodiments, the administering results in depletion of scurfin positive T cells. In some embodiments, the administering results in depletion of FoxP3+ cells (forkhead box 3 positive cells).

In various embodiments, the administering results in an expansion of cytotoxic T cells. In various embodiments, the administering results in agonism of a regulatory nuclear receptor wherein the regulatory nuclear receptor is NR0B2 (nuclear receptor subfamily 0, group B, member 2).

In some embodiments, the cancer is breast cancer, pancreatic cancer, lung cancer, melanoma, skin cancer, hematopoietic cancer, leukemia, lymphoma, colon cancer, rectal cancer, kidney cancer, renal cancer, urinary bladder cancer, oral cavity cancer, pharynx cancer, thyroid cancer, head and neck cancer, brain cancer, bone cancer, splenic cancer bladder cancer, esophageal adenocarcinoma, glioblastoma, hepatocellular carcinoma, urothelial cancer, muscle cancer, sarcoma, rhabdomyosarcoma, ovarian cancer, cervical cancer, uterine cancer, or prostate cancer.

In various embodiments, the compound activates a transcription factor. In various embodiments, the transcription factor is farnesoid X receptor (FXR), estrogen receptor alpha (ERα), estrogen receptor beta (ERβ), liver x receptor alpha (LXRα), liver x receptor beta (LXRβ), liver receptor homolog 1 (LRH-1), or nuclear factor-κB (NF-κB).

In various embodiments, the treating further comprises immune therapy. In various embodiments, the immune therapy comprises administering a second anti-cancer agent such as an interleukin, a cytokine, a chemokine, an immunomodulatory imide drug, CAR-T cells, TCR therapy, a monoclonal antibody, a cancer vaccine, a checkpoint inhibitor, or combinations thereof.

In some embodiments, the method further comprises administering a chemotherapeutic agent, radiation therapy, hormone therapy, performing surgery, or a combination thereof. In some embodiments, the method comprises inhibiting formation of solid tumors or decreasing the size of solid tumors. In some embodiments, the method comprises inhibiting formation of metastatic lesions or decreasing the size of metastatic lesions. In some embodiments, the method comprises slowing growth of solid tumors or metastatic lesions.

This disclosure additionally provides a method of inhibiting the progression of a cancer, the method which comprises contacting regulatory nuclear receptor NR0B2 (nuclear receptor subfamily 0, group B, member 2) and an effective amount of an NR0B2 agonist, wherein the regulatory nuclear receptor that is agonized by the agonist is present within the microenvironment of the cancer and progression of the cancer is thereby inhibited. In some embodiments, the agonist increases progression free survival time. In other embodiments, the agonist improves a clinical response to cancer treatment.

In various embodiments, the NR0B2 agonist is a compound of Formulas I, II, or III. In some embodiments, the cancer is a cancer disclosed herein. In some embodiments, the cancer is breast cancer or triple negative breast cancer. In some embodiments, the progression of cancer is further inhibited with a second anti-cancer agent. In some embodiments, the second anti-cancer agent is an immune checkpoint inhibitor such as pembrolizumab (Keytruda), ipilimumab (Yervoy), nivolumab (Opdivo) and atezolizumab (Tecentriq).

This disclosure also provides a method of treating a bacterial infection, a viral infection, or a fungal infection comprising administering to a subject in need thereof an effective amount of a compound or composition of a compound of Formulas I, II, or III. In some embodiments, the compound of Formula I is a compound wherein R¹ is (C₁-C₈)alkyl when R² and R³ are both ethyl and R⁴ is H. In other embodiments, the compound of Formula I is a compound wherein R¹, R², R³, and R⁴ are each not H.

In various embodiments, the compound is methyl 5-(N,N-diethylsulfamoyl)-3-hydroxy-2-naphthoate (DSHN-OMe), or a pharmaceutically acceptable salt thereof.

### Re-Education of Myeloid Immune Cells to Reduce Regulatory T Cell Expansion and Impede Breast Cancer Progression.

Although undoubtedly multifactorial, one major obstacle to ICB is the highly immune-suppressive microenvironment of breast tumors—a phenomenon that is strongly maintained by myeloid immune cells, including macrophages, and regulatory T cells (T_{regs}). Importantly however, myeloid cells are also critical for antigen presentation and a robust anti-tumor response. Therefore, it is now appreciated that rather than just eliminating or inhibiting myeloid cells, strategies are required to ‘re-educate’ myeloid cells away from being pro-tumorigenic and towards being anti-tumorigenic.

Clinical observations have identified an association between hypercholesterolemia and breast cancer recurrence. On the other hand, large retrospective studies have now shown that patients taking cholesterol lowering medication (inhibitors of 2-hydroxy-3-methylglutaryl coenzyme A reductase; HMGCR; statins) demonstrate a significantly increased time to breast cancer recurrence. Recent studies found that statins were associated with improved breast cancer specific and overall survival in TNBC patients (J Cancer 8, 2026-2032 (2017)). Collectively, these retrospective studies clearly highlight the direct clinical correlations between cholesterol and recurrence with metastatic disease. Our preclinical work has demonstrated that mice on a high cholesterol diet have increased tumor growth and metastatic burden (Nat Commun 8, 864 (2017)). Interestingly, it was found that a metabolite of cholesterol, 27-hydroxycholesterol (27HC), worked through myeloid cells to dramatically impair T cell expansion and function, and release extracellular vesicles that promote mammary cancer tumor growth and metastasis. The effects of 27HC have been attributed to its ability to selectively modulate both the estrogen receptors (ERs) and liver x receptors (LXRs), major regulators of cholesterol homeostasis (Cancer letters 493, 266-283 (2020)).

Since myeloid cells appeared particularly susceptible to perturbations in cholesterol homeostasis, we explored whether other factors in this regulatory cascade might provide useful therapeutic targets, with a focus on nuclear receptors given that they are amenable to small molecule targeting (J Steroid Biochem Mol Biol 191, 105364 (2019)). Here, we identify a regulatory nuclear receptor, NR0B2 (small heterodimer partner) whose tumoral expression were associated with improved survival. NR0B2 within the liver is known to inhibit LXRs, but it also has unique attributes in myeloid cells resulting in T cell expansion away from T_{regs} (Endocrinology 160, 1573-1589 (2019)). We show these myeloid-modulatory effects can be leveraged to promote a pro-immune and anti-cancer response.

### Results.

NR0B2 is expressed within breast tumors and is associated with increased recurrence free survival. When screening for proteins involved in cholesterol homeostasis that were also (1) potentially druggable and (2) implicated in breast cancer survival, we identified NR0B2. We initially surveyed mRNA expression of NR0B2 in different subtypes of breast cancer tumors and found that it was expressed consistently lower in the Basal and Normal breast cancer subtypes compared to others. NR0B2 had slightly higher expression in ERα+ tumors compared to ERα-ones. mRNA expression of NR0B2 across different breast cancer cell lines was relatively low compared to a liver cancer line. Likewise, NR0B2 in murine mammary cancer lines was low compared to liver tissue, with myeloid cell lines having slightly higher expression.

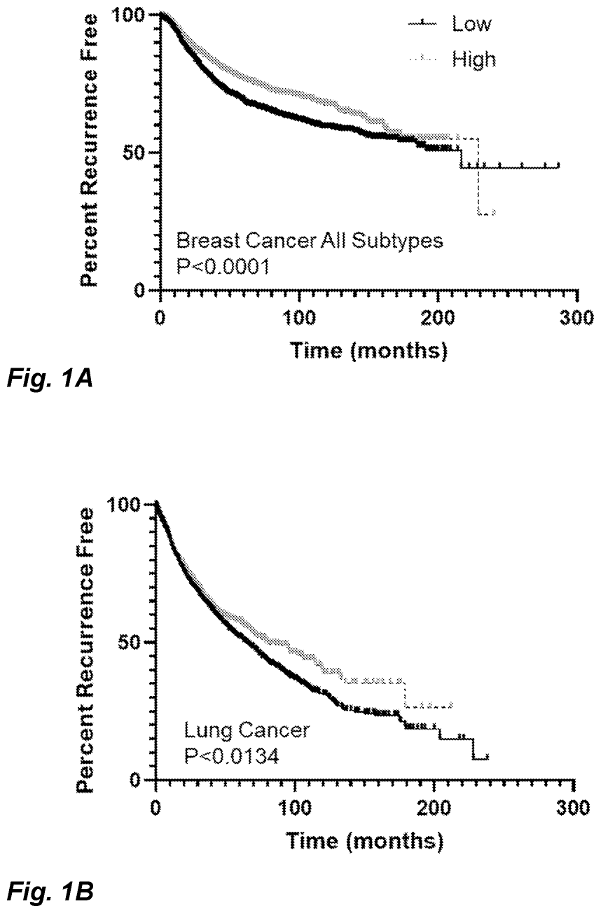

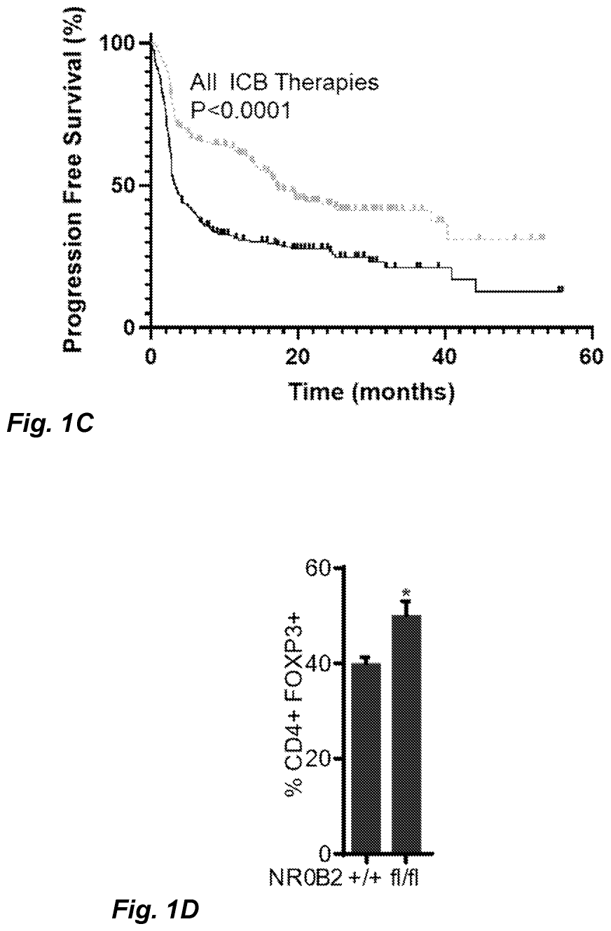

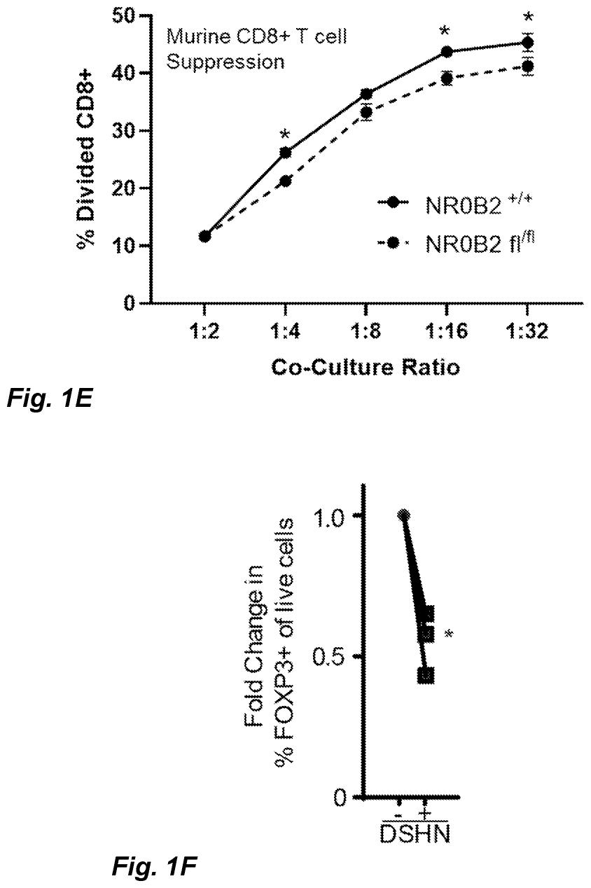

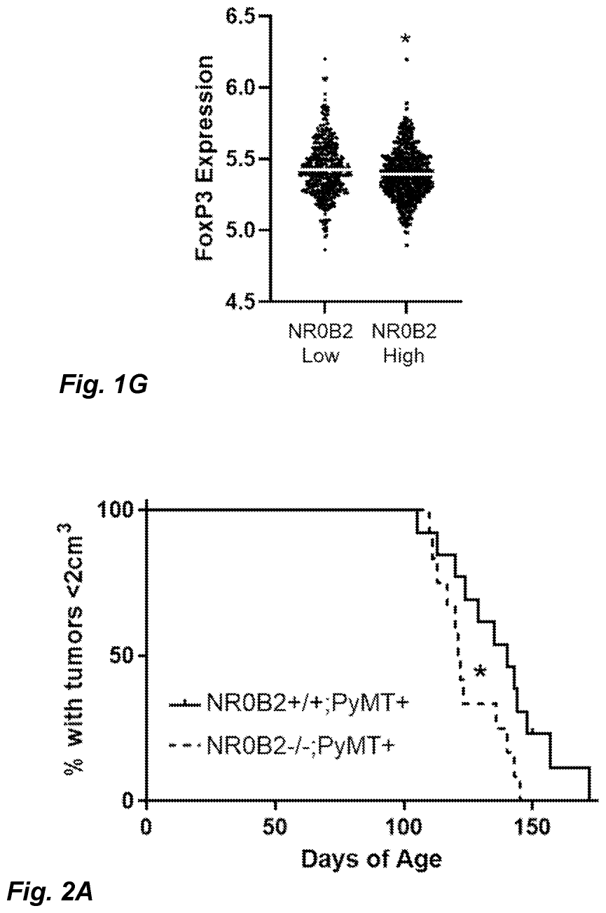

Despite its lower expression in breast cancer cells compared to liver, elevated NR0B2 expression within human breast tumors was associated with increased recurrence free survival (FIG. 1A). This was apparent when all breast cancer subtypes were considered, or when Luminal A, B, or Basal were considered independently. The HER2 subtype was underpowered to draw firm conclusions. We also found that lung cancer tumors with increased NR0B2 were associated with improved survival (FIG. 1B), an important finding given the prevalence of lung cancer, and that the lung is a clinically important metastatic site for breast cancer. Several other cancers that were assessed also showed an association between elevated NR0B2 expression and overall survival, providing a clue that commonalities within the host microenvironment may be responsible for any potential effects of NR0B2 (significant associations found in bladder cancer, renal clear cell carcinoma and lung adenocarcinoma). Even more strikingly, there is a strong association between NR0B2 expression and both increased overall and progression free survival in patients treated with ICB, a trend that holds when only those patients treated with either αPD-1, αPD-L1 or αCTLA4 are considered (pan-cancer analysis, FIG. 1C).

Protective attributes of NR0B2 are likely due to its expression in myeloid cells. Neither overexpression nor siRNA mediated knockdown of NR0B2 had a significant effect on proliferation on various breast cancer cell lines, despite eliciting expected changes in target gene expression. Likewise, treatment with the NR0B2 agonist DSHN (Mol Cancer Ther 15, 2294-2301 (2016)) had no effect on proliferation. These approaches to modulate NR0B2 activity also failed to impact cellular migration. This would suggest that the protective effects of NR0B2 observed in FIG. 1 were mediated by cells within the microenvironment, not the cancer cells themselves. scRNA-seq analysis of normal breast tissue indicates that the only cell types that express NR0B2 to appreciable levels are breast glandular cells and macrophages, although fibroblasts and breast myoepithelial cells also had some expression (data from The Human Protein Atlas). Among human PBMCs, various subtypes of dendritic cells (DCs) and monocyte populations had elevated expression of NR0B2. Our findings that NR0B2 did not impact breast cancer cell proliferation, coupled with previous data indicating that bone marrow and myeloid cells have high expression of NR0B2 compared to other tissues (Endocrinology 160, 1573-1589 (2019)), suggest that the favorable prognostic association with NR0B2 in breast tumors (FIG. 1A, 1C) was potentially mediated through myeloid immune cells.

When evaluating different myeloid populations, we observed that NR0B2 expression was downregulated in cells associated with an immune-suppressive phenotype such as M2 polarized macrophages, and myeloid derived suppressor cells. Furthermore, dendritic cells isolated from tumor bearing mice also had decreased NR0B2. Collectively, these observations suggest that NR0B2 within myeloid cells may exert anti-tumoral properties and is downregulated as part of the immune-suppressive program found in tumors. Importantly in this regard, we have previously shown that NR0B2 within macrophages was able to skew T cell expansion away from the highly immune-suppressive, regulatory T cell type (T_{reg}). Tumoral infiltration of T_{regs} is a poor prognostic and implicated in resistance to most therapies. We confirmed that genetic loss of NR0B2 in murine bone marrow derived macrophages (BMDMs, from NR0B2^{fl/fl}; LysMcre⁺ mice) resulted in increased T_{reg} expansion (FIG. 1D). Furthermore, T cells expanded in co-culture with NR0B2^{fl/fl}; LysMcre⁺ BMDMs inhibited the normal expansion of CD8⁺ T cells, demonstrating their suppressive nature.

To demonstrate that this axis is conserved in humans, we made use of cells from healthy volunteers and found that treatment of PBMC-derived-DCs with a small molecule agonist of NR0B2, DSHN, resulted in decreased T_{reg} expansion (FIG. 1F). To gain further insight into the relevance of these findings in human disease, we assessed the METABRIC dataset and found an inverse correlation between NR0B2 and FoxP3 expression, FoxP3 being a marker of T_{regs} (FIG. 1G). We then obtained serial sections from 93 human breast tumors and stained for NR0B2, FoxP3 and CD8. Extensive optimization failed to find an antibody specific or suitable for protein staining of NR0B2, so we instead used an in situ hybridization approach for this target. Sections were counter-stained with cytokeratin allowing us to evaluate differences between tumor nests and stromal regions. CD8 cells were more abundant in the tumor stroma of HER2+ cases, but at similar concentrations within the tumor itself compared to other subtypes. FoxP3 cells were lower in ERα+/PR+ cases compared to HER2⁺ or TNBC cases, regardless of tumor or stroma. NR0B2 within the tumor or stroma was significantly more prevalent in ERα+/PR+ cases compared to HER2⁺ or TNBC cases. This is in congruence with the METABRIC data indicating that ERα⁺ tumors had elevated NR0B2 expression compared to ERα⁻ ones. In general, CD8 and FoxP3 expression was correlated within the tumoral and stromal areas (Table 1).

TABLE 1
Tumor versus stroma correlations.

| | | | |
|---|---|---|---|
| Tumor % CD8+ | Stroma % CD8+ | Tumor % FoxP3+ | Stroma % FoxP3+ |

Spearman correlation and P value for CD8 and FoxP3 cells in tumor and stroma: ER/PR (n = 35)

| Tumor % CD8+ | 1.00 | | | | | |
|---|---|---|---|---|---|---|
| Stroma % CD8+ | 0.538 | (0.001) | 1.00 | | | |
| Tumor % FoxP3+ | 0.146 | (0.401) | 0.125 | (0.476) | 1.00 | |
| Stroma % FoxP3+ | −0.192 | (0.270) | 0.263 | (0.127) | 0.560 (<0.001) | 1.00 |

Spearman correlation and P value for CD8 and FoxP3 cells in tumor and stroma: HER2 (n = 30)

| Tumor % CD8+ | 1.00 | | | | | |
|---|---|---|---|---|---|---|
| Stroma % CD8+ | 0.721 | (<0.0001) | 1.00 | | | |
| Tumor % FoxP3+ | 0.220 | (0.242) | 0.177 | (0.349) | 1.00 | |
| Stroma % FoxP3+ | 0.198 | (0.294) | 0.263 | (0.161) | 0.609 (<0.001) | 1.00 |

Spearman correlation and P value for CD8 and FoxP3 cells in tumor and stroma: TNBC (n = 28)

| Tumor % CD8+ | 1.00 | | | | | |
|---|---|---|---|---|---|---|
| Stroma % CD8+ | 0.885 | (<0.0001) | 1.00 | | | |
| Tumor % FoxP3+ | 0.705 | (<0.001) | 0.688 | (0.0001) | 1.00 | |
| Stroma % FoxP3+ | 0.612 | (<0.001) | 0.662 | (0.001) | 0.702 (<0.0001) | 1.00 |

In strong support of our preclinical findings, NR0B2 expression was inversely correlated with FoxP3 (Table 2, Table 3). When assessing all tumor types together, this inverse correlation was observed whether just the tumor nest or stromal sections were considered. However, when assessing specific tumor subtypes, only TNBC cases had the inverse correlation within the stromal compartment, while all three subtypes exhibited the inverse correlation within the tumor area itself (Table 2, Table 3).

TABLE 2
Human serial section staining correlation with NR0B2.

| | Mean probes per tumor area |
|---|---|
| | [Spearman correlation (P value)] |
| Tumor % CD8+ | 0.039 (0.708) |
| Stroma % CD8+ | −0.083 (0.427) |
| Tumor % FoxP3+ | −0.371 (<0.001) |
| Stroma % FoxP3+ | −0.344 (<0.001) |

There is an inverse correlation between NR0B2 and FoxP3 in a French cohort of breast cancer samples. Breast tumors were serially sectioned and stained with NR0B2 (in situ hybridization), FoxP3 (IHC) or CD8 (IHC). Sections were counterstained with cytokeratin to differentiate between tumoral and stromal regions. Representative counterstained section above table indicating Spearman correlation and P value (N=93). * indicates P<0.05 by indicated statistical test. Extended analysis in Table 3.

TABLE 3
NR0B2-FoxP3 correlations.

| | | | |
|---|---|---|---|
| Mean probes per | Mean probe area | Mean probes | Percent tumor cells |
| tumor cell | per tumor cell area | per tumor area | within >0 probes |

ALL CASES: Spearman correlation and P value of NR0B2 probe metrics vs. CD8 and FoxP3 cells

| | | | | |
|---|---|---|---|---|
| Tumor % CD8+ | −0.001 (0.939) | −0.033 (0.751) | 0.039 (0.708) | −0.005 (0.965) |
| Stroma % CD8+ | −0.115 (0.272) | −0.131 (0.211) | −0.083 (0.427) | −0.114 (0.276) |
| Tumor % FoxP3+ | −0.425 (<0.0001) | −0.427 (<0.0001) | −0.371 (<0.001) | −0.405 (<0.0001) |
| Stroma % FoxP3+ | −0.353 (<0.001) | −0.365 (<0.001) | −0.344 (<0.001) | −0.333 (0.001) |

ER/PR: Spearman correlation and P value of NR0B2 probe metrics vs. CD8 and FoxP3 cells

| | | | | |
|---|---|---|---|---|
| Tumor % CD8+ | −0.043 (0.81) | −0.029 (0.87) | −0.016 (0.93) | −0.049 (0.78) |
| Stroma % CD8+ | −0.020 (0.91) | 0.030 (0.96) | 0.010 (0.96) | −0.041 (0.81) |
| Tumor % FoxP3+ | −0.287 (0.09) | −0.263 (0.13) | −0.283 (0.10) | −0.258 (0.13) |
| Stroma % FoxP3+ | −0.019 (0.91) | −0.003 (0.98) | −0.006 (0.97) | −0.007 (0.97) |

HER2: Spearman correlation and P value of NR0B2 probe metrics vs. CD8 and FoxP3 cells

| | | | | |
|---|---|---|---|---|
| Tumor % CD8+ | 0.252 (0.18) | −0.236 (0.42) | 0.420 (0.02) | 0.318 (0.09) |
| Stroma % CD8+ | 0.230 (0.22) | −0.225 (0.22) | 0.291 (0.12) | 0.256 (0.17) |
| Tumor % FoxP3+ | −0.441 (0.01) | −0.453 (0.01) | −0.306 (0.10) | −0.371 (0.04) |
| Stroma % FoxP3+ | −0.184 (0.33) | −0.208 (0.27) | −0.186 (0.32) | −0.079 (0.68) |

TNBC: Spearman correlation and P value of NR0B2 probe metrics vs. CD8 and FoxP3 cells

| | | | | |
|---|---|---|---|---|
| Tumor % CD8+ | −0.081 (0.68) | −0.159 (0.42) | −0.070 (0.73) | −0.117 (0.55) |
| Stroma % CD8+ | −0.269 (0.17) | −0.309 (0.11) | −0.191 (0.33) | −0.280 (0.15) |
| Tumor % FoxP3+ | −0.334 (0.08) | −0.323 (0.09) | −0.258 (0.19) | −0.349 (0.07) |
| Stroma % FoxP3+ | −0.428 (0.02) | −0.477 (0.01) | −0.348 (0.07) | −0.442 (0.02) |

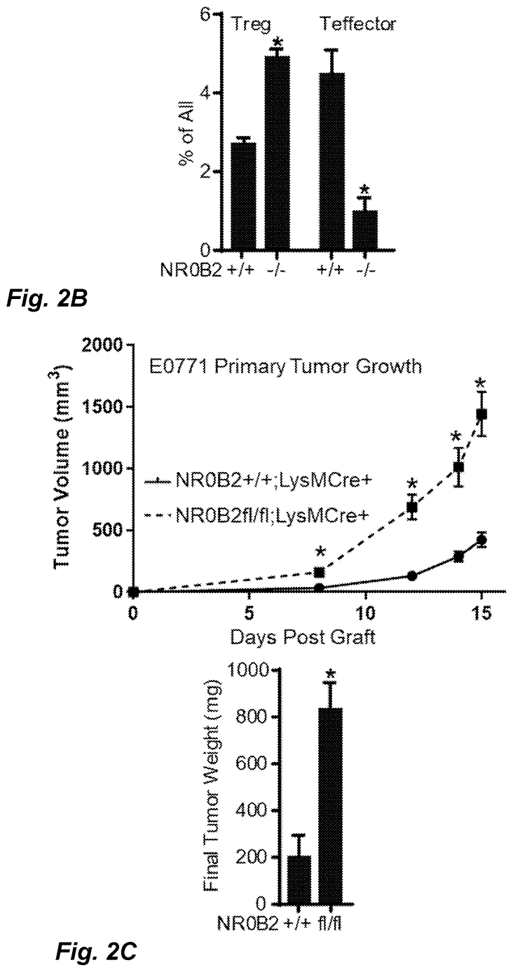

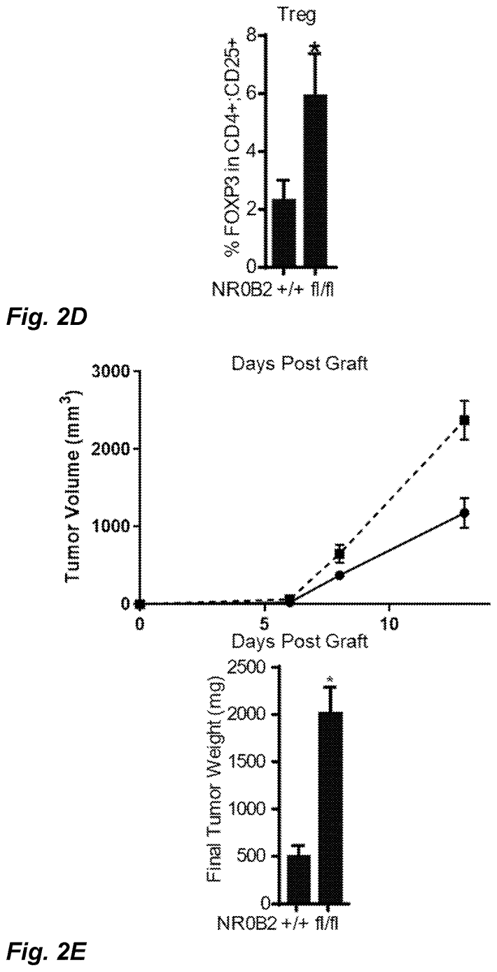

Myeloid cell loss of NR0B2 promotes tumor growth and metastasis in preclinical models. Given the strong correlational data observed in human patients (FIG. 1), and our finding that NR0B2 in myeloid cells skews T cell expansion away from T_{regs}, it was important to directly test whether NR0B2 altered tumor pathophysiology. Germline NR0B2^{-/-} mice were bred with MMTV-PyMT mice, that develop mammary tumors with progression reflective of human disease. Intriguingly, total tumor burden reached an endpoint size sooner in mice lacking NR0B2 compared to wildtype controls (FIG. 2A). Importantly, tumors from mice lacking NR0B2 had an increased T_{reg} and decreased T_{eff} infiltrate as determined by flow cytometry of digested tumors (FIG. 2B). Interestingly, the infiltrate of both M1- and M2-like macrophages was also increased in NR0B2^{-/-} tumors.

In order to evaluate the myeloid cell specific contributions of NR0B2, we generated NR0B2^{fl/fl}; LysMcre⁺ mice. Syngeneic grafts with mammary tumor line E0771 grew faster in NR0B2^{fl/fl}; LysMcre⁺ compared to control mice NR0B2^{+/+}; LysMcre⁺ (FIG. 2C). Similar to the NR0B2^{-/-}; PyMT⁺ tumors, E0771 tumors from myeloid cell specific knockout mice had increased T_{regs}, which were also observed in lung metastatic lesions (FIG. 2D). They also had increased myeloid cell infiltrate in general, although among these cells there was no difference in MHCII positivity (a metric of the capacity to present antigen) but did have increased positivity for the immune checkpoint PD-L1. This observation is likely significant given that high checkpoint levels are one mechanism for immune suppression. The difference in tumor growth was consistent across two other syngeneic cancer models (melanoma and lung), suggesting that this axis is conserved between tumor microenvironments of different solid tumors (FIG. 2E, for the Lewis lung model a sub-optimal number of cells were grafted resulting in poor tumor growth in wildtype mice and allowing for increased time for an immune-mediated response).

Tool compound, DSHN, an agonist of NR0B2 reduces breast tumor progression in preclinical models. T_{regs} have long been known to promote tumor progression and hinder the efficacy of various treatments, however, the main differentiating protein, FoxP3 has proven challenging to target therapeutically. Therefore, our finding that NR0B2 within myeloid cells results in skewed T_{reg} expansion presents an opportunity to alter T_{reg} abundance indirectly. Being a nuclear receptor, NR0B2 is highly amenable to small molecule modulation. One such compound, DSHN, has previously been described as an NR0B2 agonist.

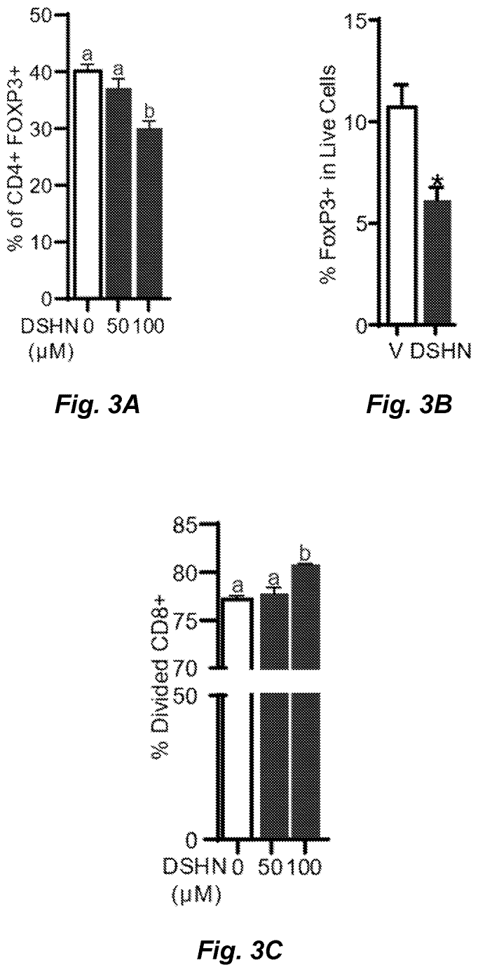

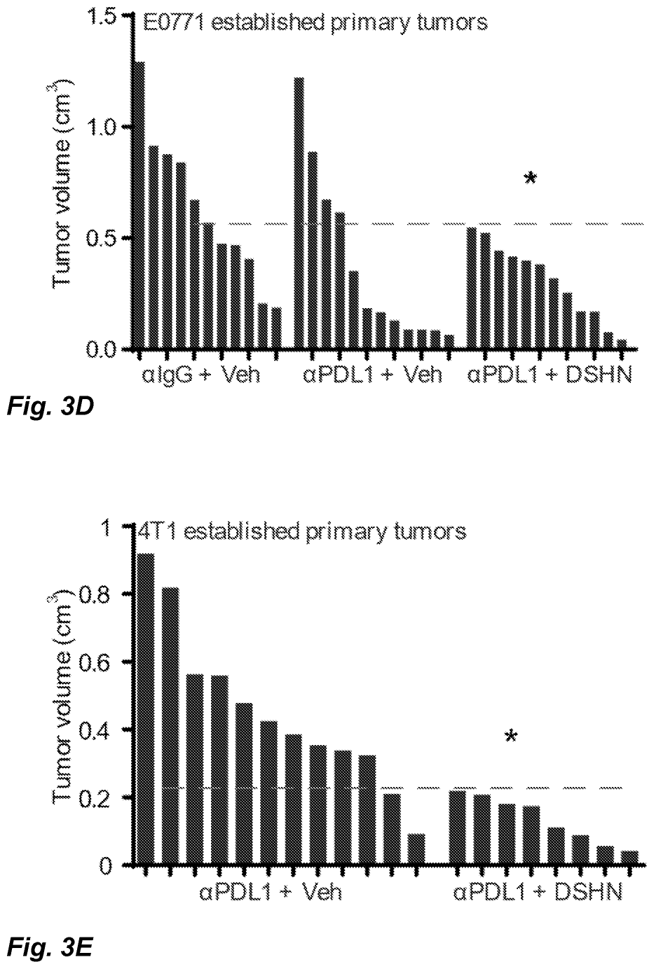

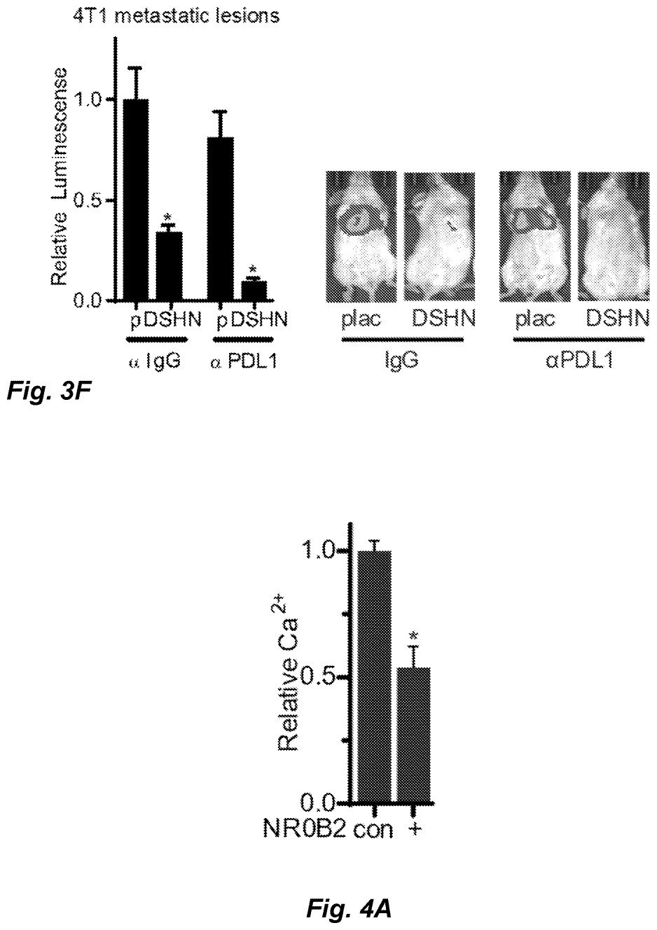

Initial characterization found that BMDMs treated with DSHN reduced subsequent T_{reg} expansion in a dose related manner (FIG. 3A). Since myeloid cells are thought to have been uniquely polarized within the tumor microenvironment, and in order to determine whether DSHN could overcome this, we isolated DCs (CD11B⁺; CD11C⁺) from MMTV-PyMT tumors. When these DCs were cultured in the presence of DSHN followed by LPS and IFNγ, they had decreased PD-L1 positivity, expectedly, the opposite to what was observed when NR0B2 was knocked out. Importantly, they also had increased viability compared to vehicle treated DCs from the same tumor (FIG. 3B left panel). When co-cultured with CD4⁺ T cells, DSHN-treated DCs resulted in a decreased expansion of T_{reg} cells, indicating that this agonist may be useful in re-educating the myeloid tumor microenvironment (FIG. 3B, right panel). Cd4⁺ T cells expanded in co-culture with DSHN pre-treated BMDMs had decreased suppressive capacity in terms of secondary CD8+ expansion (FIG. 3C). Functionally, DSHN was able to reduce T_{reg} abundance in an in vivo model of immune tolerance to a chronic antigen, where T_{regs} are typically increased. Collectively, these results were consistent with those found in human cells as described earlier (FIG. 1D).

We then evaluated DSHN in a model where a low, suboptimal number of E0771-luc cells were grafted orthotopically. Since NR0B2 was also implicated in regulating myeloid cell expression of PD-L1, we investigated the effect of acute DSHN combined with anti-PD-L1 (αPDL1) treatment on subsequent outgrowth of E0771 tumors. Treatment was ceased after 18 days and mice were monitored through time. Intriguingly, one DSHN-only treated mouse lived for more than 400 days post graft with no palpable tumor forming, while DSHN combined with αPDL1 significantly increased survival time compared to placebo treated mice. We next assessed the effects of DSHN combined with αPDL1 on growth of primary tumors (optimal number of cells used to establish graft). Treatment of animals bearing established E0771 or 4T1 tumors with DSHN combined with αPDL1 resulted in decreased growth (FIG. 3D, 3E). We then evaluated the ability of DSHN to impact established metastatic disease—a stage that is refractory to standard of care therapy and immune checkpoint blockade. As expected, αPDL1 did not significantly impact metastatic outgrowth in this model (FIG. 3F). However, DSHN alone slowed the growth of metastatic lesions. Furthermore, combining DSHN with αPDL1 reduced outgrowth even further than DSHN alone, indicating that simultaneously targeting the innate and adaptive arms of the immune system will be beneficial for the treatment of immune-suppressive tumors such as breast cancer. In summary, the tool compound DSHN provides proof of principle that the NR0B2-myeloid cell-T_{reg} axis can be targeted for the treatment of breast cancer.

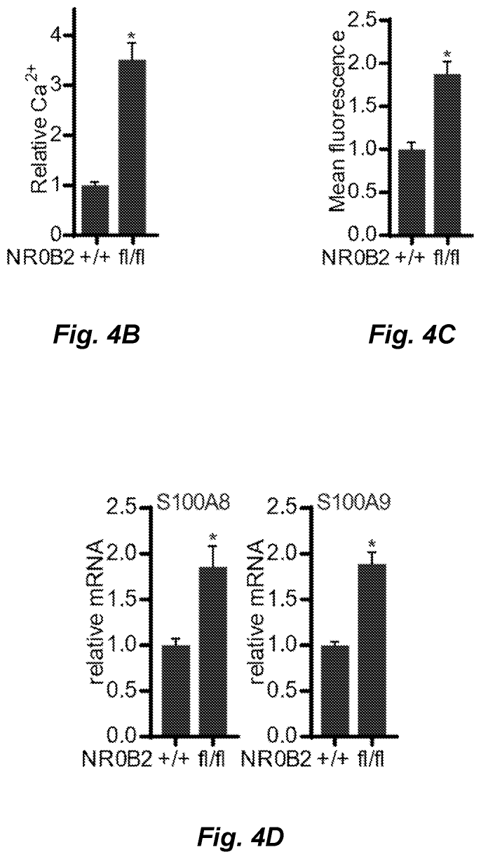

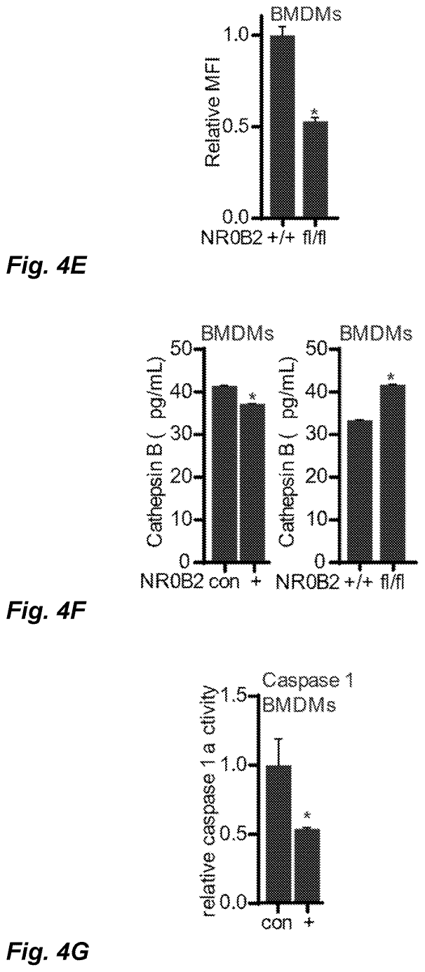

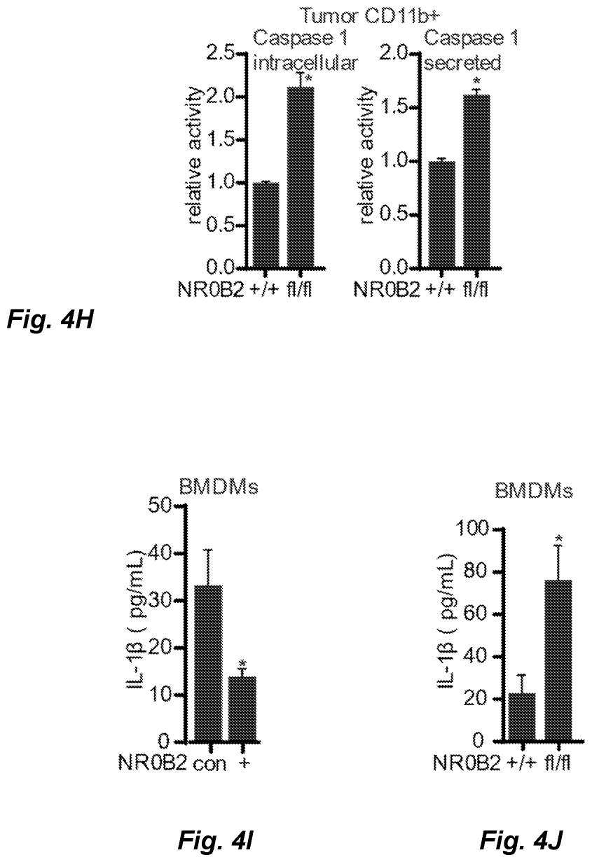

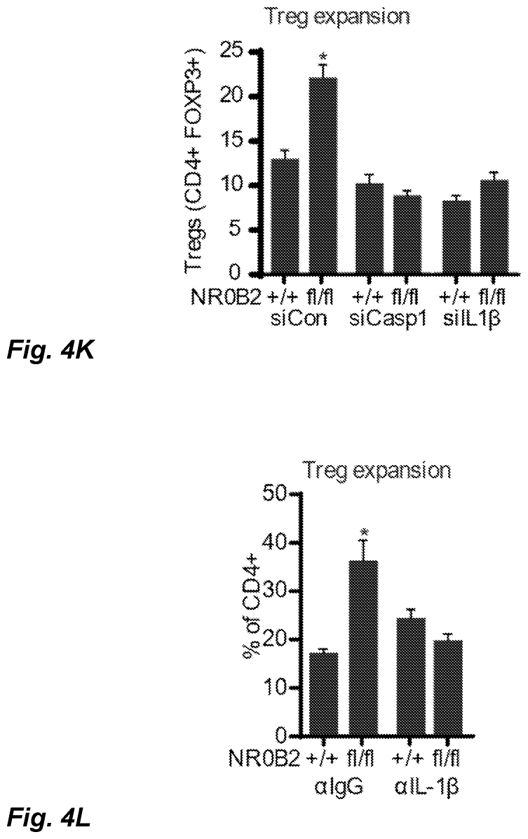

Inflammasome implicated in myeloid cell NR0B2-T_{reg} axis. In order to gain insight into the potential mechanisms of action, we compared mRNA profiles from (1) loss of NR0B2 in PyMT tumors (from FIG. 2A) (2) in vitro BMDMs treated with the agonist DSHN, and (3) 4T1 metastatic lungs from mice treated with DSHN. Since NR0B2 is known to inhibit LXR, we also included groups of BMDMs treated with the synthetic LXR agonist GW3965, and both GW3965 and DSHN. A signature of upregulated genes in BMDMs treated with DSHN versus vehicle was found to be correlated with survival in breast cancer patients. In all three data sets, we observed that several genes associated with the inflammasome were altered. Given previous reports that the inflammasome can drive T_{reg} differentiation and expansion via myeloid cells, we interrogated aspects of the inflammasome from upstream regulation to downstream execution (overview cartoon in top panel of FIG. 4). Intracellular Ca²⁺ was decreased when NR0B2 was overexpressed in naïve BMDMs or DCs treated with ionomycin or PMA, and vice versa when NR0B2 was knocked out (FIG. 4A, 4B). Reactive oxygen species (ROS) were increased in the absence of NR0B2 (FIG. 4C). S100A8 and S100A9 transcripts were altered by NR0B2 in CD11B+ myeloid cells isolated from tumors, or BMDMs co-cultured with E0771 cancer cells (FIG. 4D). Lysosome stability (ie: increased pH) was decreased when NR0B2 was knocked out in BMDMs or DCs (FIG. 4E). Cathepsin B mRNA was regulated when NR0B2 was manipulated in BMDMs or CD11B+ cells from tumors. Likewise, secreted cathepsin B protein was decreased when NR0B2 was overexpressed, or increased when NR0B2 was knocked out in BMDMs, CD11B+ cells isolated from tumors or DCs (FIG. 4F). When NR0B2 was overexpressed, the downstream executioner of the inflammasome, caspase 1, had decreased transcript and activity (FIG. 4G), with the reverse being true in CD11B+ or CD11C+ cells from E0771 tumors grown in mice lacking myeloid NR0B2 (FIG. 4H). IL-1β mRNA was regulated in opposite ways when NR0B2 was knocked out or overexpressed, as was its secreted protein (FIG. 4I, 4J). These data indicate that NR0B2 is suppressing several different steps in the inflammasome cascade. Indeed, even when the inflammasome was activated with S100A8/9 and ionomycin, overexpression of NR0B2 blunted the induction of caspase 1, IL1β, IL18 and several downstream chemokines. IL-1β has previously been shown to promote FOXP3 expression and promote T_{reg} differentiation. Knockdown of IL-1β in BMDMs resulted in decreased T_{reg} expansion, confirming that decreased IL-1β is likely the downstream mechanism of NR0B2. Importantly, the increased T_{reg} expansion when cultured with BMDMs lacking NR0B2 was lost when BMDMs were treated with siRNA against caspase 1, siRNA against IL-1β (FIG. 4K), or an antibody to neutralize IL-1β (FIG. 4L), providing strong support for inflammasome pathway modulation being the primary mechanism by which NR0B2 influences T_{reg} expansion.

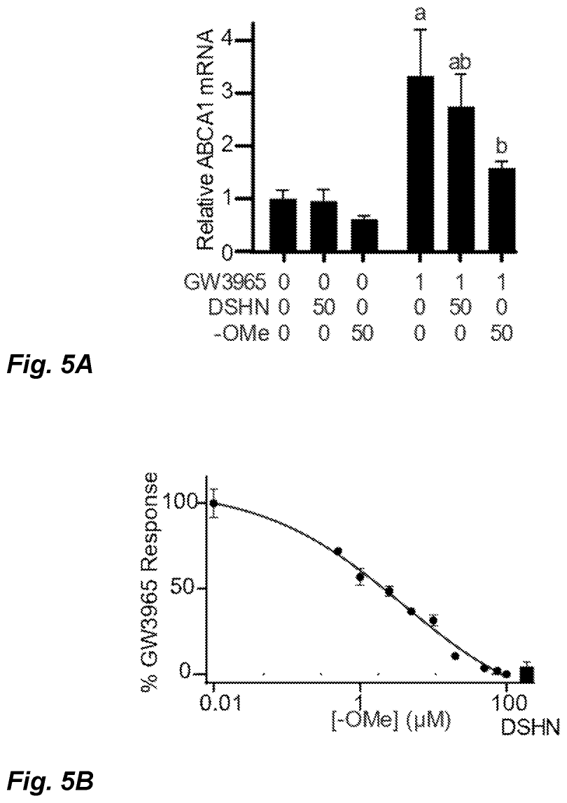

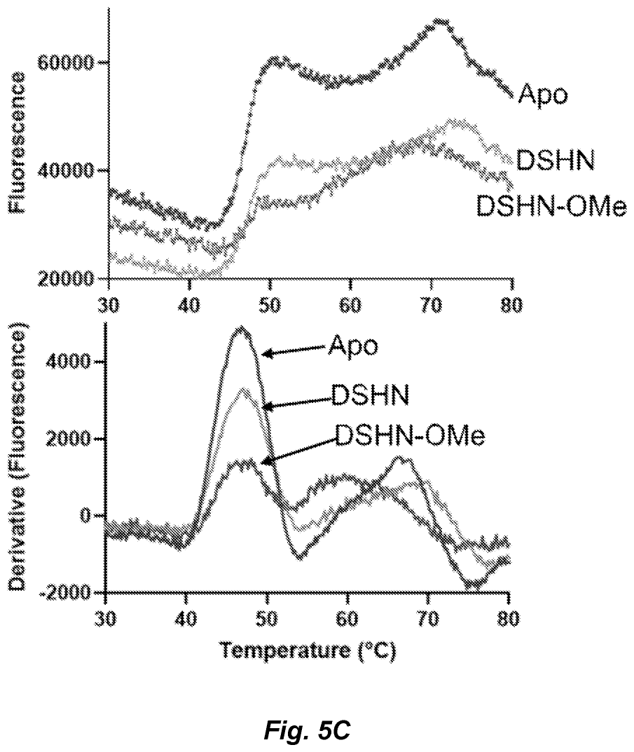

Development of a more efficacious NR0B2 agonist. While DSHN was a useful proof-of-concept small molecule, macrophages required high doses and chronic exposure for robust influences on subsequent T_{regs} expansion. Furthermore, it proved to have poor aqueous solubility, potentially limiting its in vivo translation. Therefore, we synthesized several derivatives of DSHN and screened them using a reporter assay which makes use of the endogenous cholesterol homeostatic feedback loop, where FXR activation upregulates NR0B2, which then inhibits LXR and its normal induction of ABCA1. We then followed this screen with several others with stringent cutoffs: effect on endogenous ABCA1 and IL1β expression, reduction of T_{reg} expansion in either macrophage or DC co-cultures, and low cellular toxicity (FIG. 5A, Table 4).

The methyl ester of DSHN (DSHN-OMe) emerged as the best performer across all of these endpoints (preparation and characterization described in Examples), with DSHN-OMe reducing LXR induction of ABCA1 in a dose-related manner (FIG. 5B). DSHN-OMe showed a modest shift in NR0B2 thermal melt temperature. The T_{M} for apo/unliganded was 46.74±0.22, DSHN-OMe at 46.92±0.17 and DSHN at 46.34±0.51° C. (FIG. 5C). Both molecules similarly reduced the magnitude of the transition suggesting that they are likely binding to the protein but not significantly affecting thermal stability. The ability of DSHN-OMe to attenuate the induction of ABCA1 expression by the LXR agonist GW3965 was lost when NR0B2 was knocked down in BMDMs, indicating that DSHN-OMe was working through its intended target (FIG. 5D) further suggesting NR0B2 as the target of DSHN-OMe. Similar to DSHN, DSHN-OMe showed minimal cytotoxicity in BMDMs. However, compared to DSHN, DSHN-OMe treated macrophages or DCs elicited a reduction in T_{reg} expansion with a lower dose, and had a larger biological maximum. Pretreatment of BMDMs followed by washout prior to co-culture with T cells resulted in decreased T_{reg} expansion for DSHN-OMe, but only modest reductions for DSHN, indicating that (i) the effects of DSHN-OMe are mediated through BMDMs/DCs, and (ii) DSHN-OMe either had better cell penetration or more durable effects on BMDMs compared to DSHN.

TABLE 4
Overview of screening DSHN derivatives, using strict cutoffs
with respect to DSHN for passing each metric.

| | | | | ABCA1 | IL1b | |
|---|---|---|---|---|---|---|
| | ABCA1 | Treg | | mRNA | mRNA | |
| | reporter | expansion | Treg expansion | reduction | reduction | Low |
| Compound | screen | macrophages | Dendritic Cells | (Raw Cells) | (Raw Cells) | Toxicity |
| DSHN-OMe | ✓ | ✓ | ✓ | ✓ | ✓ | ✓ |
| 5 | X | ✓ | ✓ | X | ✓ | ✓ |
| 6 | ✓ | ✓ | ✓ | X | X | X |
| 7 | ✓ | ✓ | ✓ | X | ✓ | ✓ |
| 8 | ✓ | ✓ | ✓ | X | X | ✓ |
| 9 | X | ✓ | ✓ | X | ✓ | ✓ |
| 10 | ✓ | ✓ | ✓ | ✓ | ✓ | ✓ |

Importantly, T cells resulting from expansion in co-culture with BMDMs that were pretreated with either DSHN-OMe or DSHN were less suppressive when subsequently cultured with a second set of naïve T cells, and specifically assessing CD8⁺ expansion. Conversion of a carboxylic acid to an ester is often associated with better cellular uptake. Indeed, DSHN had very poor uptake by RAW264.7 cells, while DSHN-OMe had considerable uptake at 4 h. Conversion of DSHN-OMe to DSHN was minimal over the 24 h time period of the experiment, suggesting that DSHN-OMe is likely the active modulator of NR0B2.

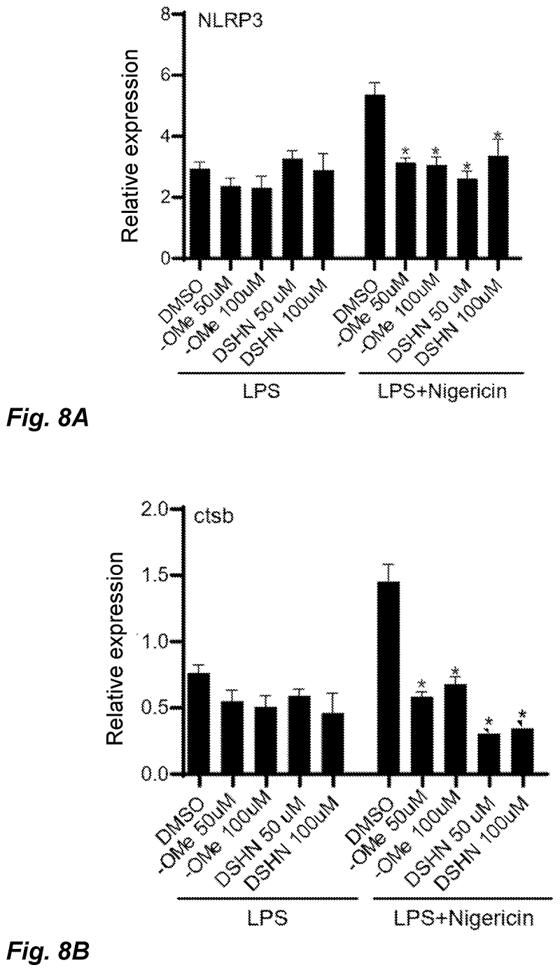

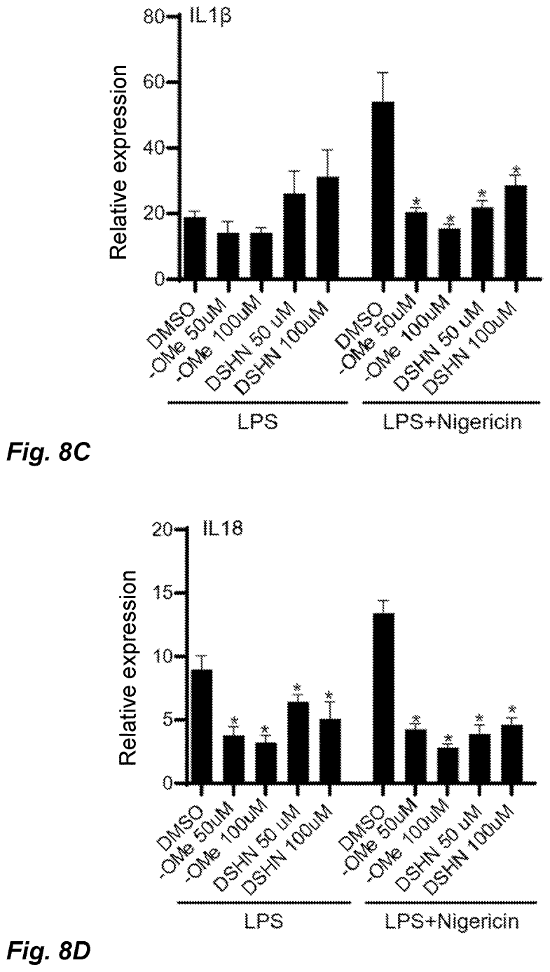

Both DSHN and DSHN-OMe were able to blunt the expression of inflammasome associated genes in the presence of both a priming signal as well as an activation signal (LPS and nigericin respectively), but not with just a priming signal, indicating that the effects of NR0B2 are primarily with respect to activation of the inflammasome (FIG. 8).

Importantly, DSHN-OMe significantly reduced the outgrowth of established 4T1 metastatic lesions as a single agent. Addition of αPDL1 did not significantly influence the effects of DSHN-OMe in this assay, although the short time frame (due to humane endpoint of placebo groups) may not have captured the contribution of checkpoint inhibition. DSHN-OMe also significantly reduced metastatic burden in mice grafted with EMT6 cells, an alternative murine mammary cancer model. Ki67 mRNA was also decreased in EMT6 metastatic lungs of DSHN-OMe treated mice, indicating a decreased number of proliferating cancer cells. As expected, metastatic lungs from DSHN-OMe treated mice had decreased FOXP3 expression, and a corresponding increase in the relative abundance of CD8⁺ cytotoxic T cells.

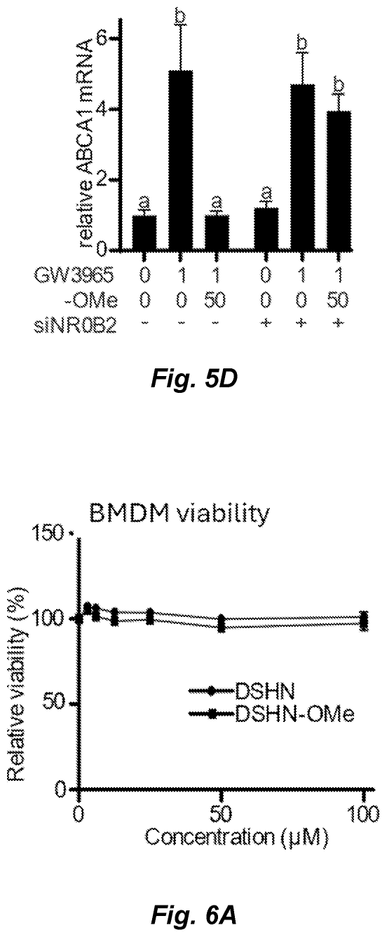

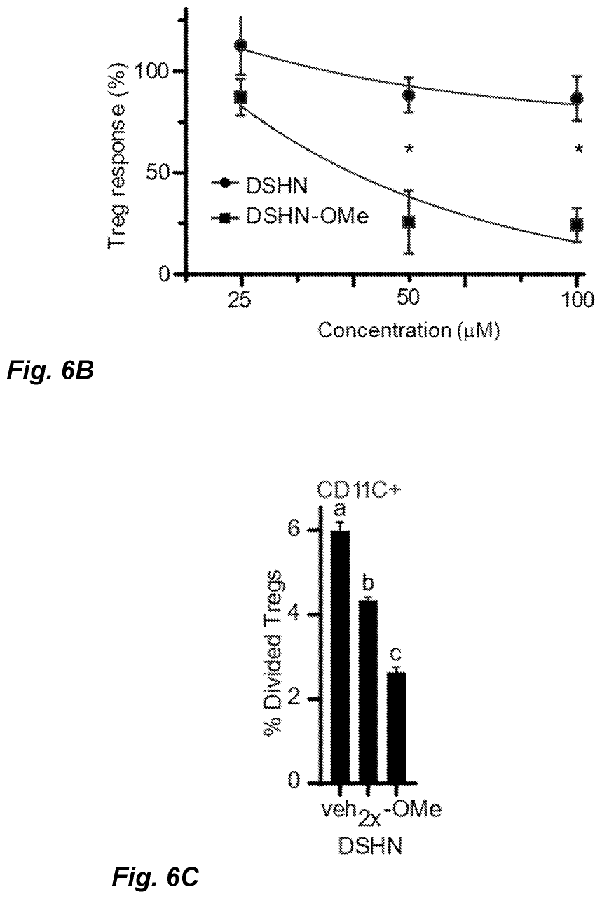

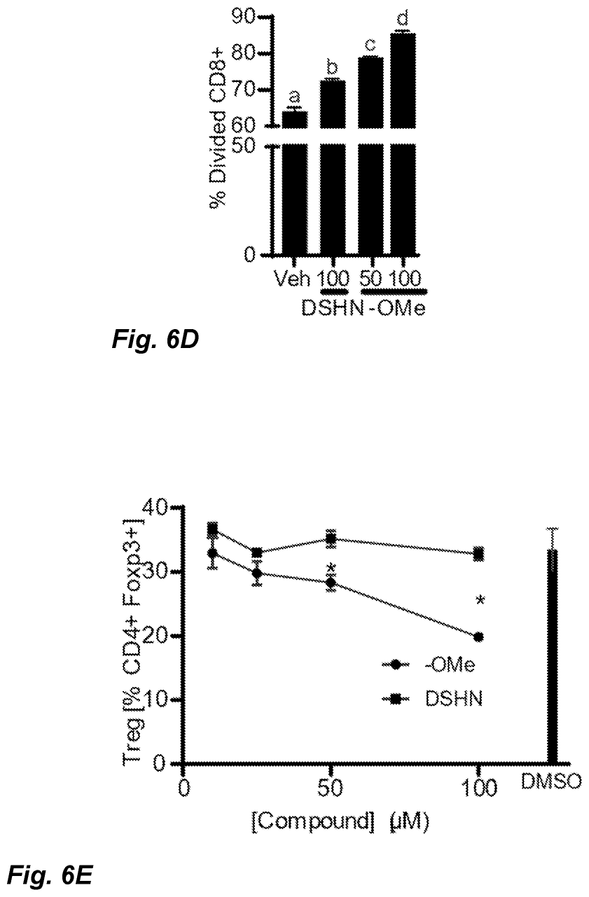

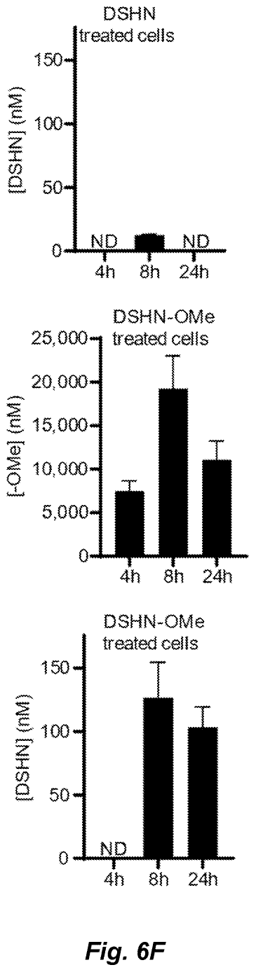

DSHN-OMe does not affect BMDM viability but significantly attenuates BMDM-regulated T_{reg} expansion. In order to better assess the potential cellular toxicity of DSHN-OMe, we treated BMDMs with increasing doses and measured their ability to reduce resazurin to resorufin (similar to the MTT viability assay). Importantly, no significant difference was observed in viability up to 100 μM for either DSHN-OMe or its parental compound, DSHN (FIG. 6A). However, compared to DSHN, DSHN-OMe treated BMDMs elicited a reduction in T_{reg} expansion with a lower dose, and had a larger biological maximum (FIG. 6B). This was also evident in splenic CD11C⁺ (DC-enriched) cells, where 50 μM DSHN-OMe treatment resulted in significantly fewer T_{regs} compared to twice the dose of DSHN (100 μM) (FIG. 6C). Importantly, T cells resulting from expansion in co-culture with BMDMs that were pretreated with either DSHN-OMe or DSHN were less suppressive when subsequently cultured with a second set of naïve T cells, specifically assessing CD8+ expansion (FIG. 6D).

In a separate experiment, BMDMs were pretreated with compound and washed with PBS several times before incubation with expanding T cells. Here, DSHN was not effective at reducing T_{reg} expansion, while DSHN-OMe was (FIG. 6E). Collectively, these data supporting the superiority of DSHN-OMe compared to its parental compound suggested that DSHN-OMe either had better cellular penetration or more durable effects on myeloid cells compared to DSHN.

Conversion of a carboxylic acid to an ester is often associated with better cellular uptake. Therefore, we explored whether the kinetics of uptake differed between the two compounds, using RAW 264.7 cells as a model for myeloid immune cells. RAW 264.7 cells were treated with 50 μM of DSHN or DSHN-OME for 4, 8 or 24 h. At the appropriate time-point cells were washed and subjected to LC-MS/MS to quantify intracellular compound concentration. Surprisingly, DSHN had very little uptake (FIG. 6F). At 4 hrs, DSHN was below detection limits. At 8 h, 12.6 nM DSHN was detected. By 24 h, no intracellular DSHN was detected. On the other hand, DSHN-OMe was detected at 7,481 nM by 4 h, increasing to 19,279 nM by 8 h, which only dropped slightly to 11,089 nM at 24 h (FIG. 6F). Interestingly, in DSHN-OMe treated cells, we were also able to detect small quantities of DSHN (<130 nM), higher than DSHN treated cells. This indicated that DSHN-OMe was being metabolized back to its parental compound upon cellular entry (FIG. 6F). Given these results, we cannot rule out the possibility that the activity of DSHN-OMe is due to conversion to DSHN, although the shear difference in intracellular concentration (>2 log) would suggest that DSHN-OMe is likely the active modulator of NR0B2 (FIG. 6F).

DSHN-OMe has significant efficacy against murine models of breast and lung cancer. Given the enhanced cellular uptake and ability to attenuate myeloid cell-modulated T_{reg} expansion, it was next important to evaluate the efficacy of DSHN-OMe in an in vivo models of mammary cancer. For these studies we made use of the syngeneic E0771 model, allowing us to also test the compound in mice lacking myeloid expression of NR0B2 (NR0B2^{fl/fl}; LysMCre⁺ mice). E0771 tumors grew at an accelerated rate in NR0B2^{fl/fl}; LysMCre⁺ mice compared to control NR0B2-replete mice (NR0B2^{+/+}; LysMCre⁺ mice FIG. 7A, 7B).

Importantly, DSHN-OMe significantly reduced E0771 tumor growth (growth through time in FIG. 7A and waterfall plot in FIG. 7B). However, the growth-reducing effects of DSHN-OMe were only observed in mice with myeloid cell expression of NR0B2, with no significant impact being observed in NR0B2^{fl/fl}; LysMCre⁺ mice (FIG. 7A, 7B). Therefore, DSHN-OMe had significant efficacy in a myeloid cell NR0B2 dependent manner, as in the observed effects were being mediated through the anticipated target.

Since T_{regs} are considered a problem common to many solid tumor types, we reasoned that DSHN-OMe should have broad efficacy. To test this, we evaluated the ability of DSHN-OMe to influence the growth of murine Lewis Lung tumors grown in the flank. Similar to our E0771 results, DSHN-OMe significantly reduced tumor growth through time compared to placebo-treated mice (FIG. 7C, 7D). Again, these anti-tumor effects were only observed in mice with replete myeloid cell expression of NR0B2 and not in NR0B2^{fl/fl}; LysMCre⁺ mice (FIG. 7C, 7D).

Metastatic breast cancer urgently requires new therapeutic approaches. Therefore, we tested the ability of DSHN-OMe to reduce the subsequent growth of established metastases. 4T1 cells were introduced intravenously and lesions allowed to establish for 3d. Since ICB has been approved for a subset of TNBC patients, we also included groups treated with αPDL1. DSHN-OMe significantly reduced the outgrowth of established 4T1 metastatic lesions as a single agent (FIG. 7E). Addition of αPDL1 did not significantly influence the effects of DSHN-OMe in this assay, although the short time frame (due to humane endpoint of placebo groups) may not have captured the contribution of checkpoint inhibition. We confirmed these findings using a different model of murine TNBC, EMT6. DSHN-OMe significantly reduced metastatic burden in EMT6-grafted mice (FIG. 7F), indicating that DSHN-OMe has broad efficacy against different mammary cancer models. Ki67 mRNA was also decreased in EMT6 metastatic lungs of DSHN-OMe treated mice, suggesting a decreased number of proliferating cancer cells (FIG. 7G). As expected, metastatic lungs from DSHN-OMe treated mice had decreased Foxp3 expression (FIG. 7H), and a corresponding increase in the relative abundance of CD8+ cytotoxic T cells (FIG. 7I). Therefore, DSHN-OMe represents an improved NR0B2 agonist with significant anti-tumor effects, both against primary tumor growth and also metastatic outgrowth.

### DISCUSSION

NR0B2 is best known as a regulator of cholesterol and bile acid homeostasis, directly binding and inhibiting LXR and LRH-1 mediated induction of cholesterol catabolism and efflux. Emerging reports indicate roles outside this axis as well as extra-hepatic roles (Nat Commun 11, 5969 (2020)). Various roles for NR0B2 in the immune system have been described. Here, we demonstrate the importance of NR0B2 in breast cancer pathophysiology. By inhibiting the inflammasome within myeloid cells, NR0B2 results in reduced T cell expansion towards T_{regs}, reduced immune suppression and ultimately, reduced tumor or metastatic outgrowth. Previous reports have indicated a role for NR0B2 in hepatic carcinomas but have largely focused on cancer-cell intrinsic roles (J Steroid Biochem Mol Biol 191, 105364 (2019)). In the models of breast cancer evaluated here, NR0B2 had no significant effects on cellular proliferation or migration indicating that these cell-intrinsic activities may be exclusive for cells of the hepatic origin. Importantly, NR0B2, as with all nuclear receptors, has a distinct ligand binding domain allowing for targeting with small molecules. Indeed, a previously reported agonist, DSHN was able to improve the efficacy of ICB, and the novel DSHN-OMe decreased metastatic outgrowth as a single agent in two models of TNBC. Several correlations using human breast cancer patient data support our preclinical observations: NR0B2 mRNA expression is associated with a good prognosis, NR0B2 expression is inversely correlated with the T_{reg} marker FOXP3 mRNA in the TCGA and FOXP3 protein in an independent cohort from France, and a signature of genes upregulated in BMDMs treated with DSHN. Furthermore, human PBMCs treated with DSHN also attenuated the expansion of T_{regs}. Therefore, this target and axis demonstrate significant potential for translation.

There is significant effort to develop new ICB targets or improve the efficacy of current ICB. Cholesterol metabolism and homeostasis is emerging as a central axis regulating immune function. Cholesterol itself plays important roles in T cell biology, in addition to LXR activation. Cholesterol metabolites shift myeloid cells to being highly immune suppressive, in part through LXR activation. However, LXR activity in myeloid immune cells appears complex, and these receptors are likely selectively modulated with different ligands exerting varying effects; this complexity providing a pharmacological opportunity. Downstream cholesterol homeostasis and bile acid signaling have also been implicated, with microbial transformed bile acids shown to alter T_{reg} expansion in a T cell-intrinsic manner. There is some evidence that these effects are mediated through the bile acid receptors Farnesoid X Receptor (FXR) or TGR5, but this still requires further investigation. NR0B2 is a direct target gene of FXR, leaving the possibility that NR0B2 may also have T cell-intrinsic effects, in addition to the myeloid cell effects described here. Regardless, the effects would be expected to be beneficial with respect to cancer therapy.

Previous work has described NR0B2 in regulating various aspects of immune function. Mice with a whole-body knockout of NR0B2 were protected from septic shock induced by LPS. Several mechanisms were proposed for the suppressive effects of NR0B2, including direct interactions with NF-κB and TRAF6, which in the case of TRAF6 led to a reduction in its ubiquitination. We have also shown that NR0B2 and NF-κB appear to colocalize, although direct binding or subsequent consequences of this interaction are less clear. The observations that NR0B2 is protective against septic shock are in contrast to what we would expect if T_{regs} were increased in the absence of NR0B2. However, the authors also note that upon LPS stimuli, macrophages upregulate NR0B2 via a Ca²⁺-dependent activation of AMPK, which would fit a model whereby NR0B2 is upregulated to reduce T_{reg} expansion and drive an immune-response. Interestingly, NR0B2 was previously found to directly interact with NLRP3 to attenuate the NLRP3 inflammasome. While this may be the case, our data indicates that to some degree NR0B2 regulates every aspect of the inflammasome investigated: Ca² flux, ROS generation, S100A8 and S100A9 mRNA regulation, lysosome stability, cathepsin B (mRNA and protein), caspase 1 activity and IL-1β (mRNA and protein). This subtle regulation across the board indicates the critical role for NR0B2 in shaping the myeloid immune response. Importantly, these observations were made in both BMDMs and DCs, suggesting that the same target (NR0B2) will have utility across all antigen presenting cells. Ultimately, the reduced inflammasome activity resulted in decreased expansion of T_{regs}, as knockdown of caspase 1 or IL-1β, or immune-neutralization of IL-1β were able to inhibit the effects of loss of NR0B2. There is indication that NR0B2 also regulates TGF-β and IL-2, two other cytokines involved in T_{reg} differentiation/expansion. Whether these are downstream effects of an attenuated inflammasome remains to be determined. Collectively, NR0B2 appears to subtly re-educate myeloid cells across several aspects of the inflammasome and cellular function. These actions subsequently decrease T_{reg} expansion, shifting the tumor microenvironment towards being less immune-suppressive.

Being a nuclear receptor, NR0B2 has a well-defined ligand binding domain, through which small molecules can bind and regulate its activity. Here, we show that a first generation NR0B2 agonist, DSHN, regulates T_{reg} expansion as expected after myeloid cell treatment, and when combined with ICB reduces the tumoral outgrowth of syngeneic mammary tumors. Using the 4T1 model of mammary metastasis, DSHN had significant effects as a single agent, perhaps reflecting the variable immune-suppressiveness in different microenvironmental niches. In vitro, DSHN required high doses and continuous exposure in order to alter T_{reg} expansion and lacked the solubility for effective translation as a therapeutic. Therefore, we developed and screened for improved derivatives, identifying DSHN-OMe as having increased efficacy in terms of T_{reg} expansion. DSHN-OMe also had superior qualities in terms of low cellular toxicity. In two models of murine mammary cancer metastasis, DSHN-OMe had robust effects as a single agent.

To identify DSHN-OMe, we made use of the endogenous cholesterol homeostasis feedback loop, whereby NR0B2 inhibits LXR. The bile acid receptor, FXR, serves to up-regulate NR0B2 when activated. Thus, in addition to targeting NR0B2 directly, it may be possible to utilize the FXR. In this regard, the semi-synthetic bile acid, obeticholic acid is FDA approved for the treatment of primary biliary cholangitis and may represent a rapidly translatable avenue for this axis. Obeticholic acid is associated with several side effects including pruritus, resulting in low patient compliance. This provides rationale for the continued development of downstream targets such as NR0B2. However, it may also be possible to use obeticholic acid acutely to re-educate the tumor microenvironment making it more susceptible to ICB. Collectively, our data provide proof of concept that mediators of downstream cholesterol homoeostasis can be leveraged for the treatment of solid tumors.

### Pharmaceutical Formulations

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze-drying techniques, which yield a powder of the active ingredient plus any additional desired ingredient present in the solution.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to enhance the properties for a given use. Thickeners such as synthetic polymers, fatty acids, fatty acid salts and esters, fatty alcohols, modified celluloses, or modified mineral materials can also be employed with liquid carriers.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day. The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

Alternatively, the compound can be conveniently administered in a unit dosage form, for example, containing 5 to 1000 mg/m², conveniently 10 to 750 mg/m², more conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The compounds described herein can be effective anti-tumor agents and have higher potency and/or reduced toxicity as compared to DSHN. Preferably, compounds of the invention are more potent and less toxic than DSHN, and/or avoid a potential site of catabolic metabolism encountered with DSHN, i.e., have a different metabolic profile than DSHN.

The invention provides therapeutic methods of treating cancer in a mammal, which involve administering to a mammal having cancer an effective amount of a compound or composition described herein. A mammal includes a primate, human, rodent, canine, feline, bovine, ovine, equine, swine, caprine, bovine and the like. Cancer refers to any various type of malignant neoplasm, for example, breast cancer, colon cancer, pancreatic cancer, melanoma and leukemia, and in general is characterized by an undesirable cellular proliferation, e.g., unregulated growth, lack of differentiation, local tissue invasion, and metastasis.

The ability of a compound of the invention to treat cancer may be determined by using assays well known to the art. For example, the design of treatment protocols, toxicity evaluation, data analysis, quantification of tumor cell kill, and the biological significance of the use of transplantable tumor screens are known. In addition, ability of a compound to treat cancer may be determined using the tests described and referenced herein.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Materials and Methods

Reagents. DSHN (purity >95%) was synthesized by Sai Life (Hyderabad, India). GW3965, PMA, Ionomycin, Obeticholic acid and GW4064 were obtained from Cayman (Ann Arbor, MI) (purity ≥98%). DSHN-OMe was synthesized in house. DSHN, Compound 6, GW3965, Nigericin, PMA, Obeticholic acid and GW4064 were dissolved in dimethyl sulfoxide (DMSO, Sigma) and stored in −20° C. Ionomycin was dissolved in ethanol and stored in −20° C. Antibodies for flow cytometry were purchased from BD Biosciences and used at a working dilution of 1:100 in FACS buffer (2% fetal bovine serum (FBS) in phosphate buffered saline (PBS)) for surface staining and 1:50 in permeabilization buffer for intracellular staining.

Survival and Correlational Analysis of human tumors. Survival analysis in FIG. 1 was performed the Kaplan-Meier Plotter webtool (www.kmplot.com/analysis) and cBioPortal (www.cbioportal.org). The Kaplan-Meier Plotter webtool uses aggregated data from GEO, EGA, and TCGA. Differentially upregulated genes between vehicle and DSHN treated BMDMs from RNA-seq analysis (fold change threshold of 2 fold, and FDR<0.01), were used to create a non-weighted signature of 22 genes. This signature was then used to probe the METABRIC dataset as obtained through cBioPortal. Tumors were then parsed into upper and lower quartiles and used to examine survival data using Kaplan-Meier analysis.

Cell lines. HepG2 cells were a gift from Sayeepriyadarshini Anakk (University of Illinois at Urbana-Champaign) and cultured in complete DMEM/F12 (supplemented with 10% Charcoal-stripped FBS (Cytiva HyClone), 1% non-essential amino acids (Corning), 1% sodium pyruvate (Corning) and 1% penicillin/streptomycin (Corning)). EMT6-luc cells were a gift from Hasan Korkaya (Augusta University) and cultured in complete DMEM/F12. E0771 and 4T1-luc cells were a gift from Mark Dewhirst (Duke University) and cultured in complete RPMI (Corning) (supplemented with 10% FBS (Cytiva HyClone), 1% non-essential amino acids (Corning), 1% sodium pyruvate (Corning) and 1% penicillin/streptomycin (Corning)). We did not culture any of these cell lines longer than 2 months after thawing the stocks or after passage 20. Cell lines were tested for mycoplasma.

Animal tissue and in vivo studies. All protocols involving animals were approved by the Institutional Animal Care and Use Committee (IACUC) at the University of Illinois Urbana-Champaign. Female wild-type C₅₇BL/6 and BALB/C mice were purchased from Charles River Laboratory. Mice were 8-12 weeks old at the start of the experiment. Founder OT-II mice were purchased from Jackson Laboratory and bred in-house. Founder NR0B2f/fl mice were a kind gift from John Auwerx and Kristina Shoonjans (Ecole Polytechnique de Lausanne). The mice were matched for age in each experiment.

Preparation of Bone Marrow-Derived Macrophages (BMDMs) and Dendritic Cells. Bone marrow cells were collected from mouse tibia and femur. Cells were passed through 70-micron cell strainer and subsequently cultured 10 mL complete RPMI media supplemented with 20 ng/mL of recombinant murine M-CSF (576406; BioLegend, 315-02 PeproTech). On day 3, additional 5 ml of the complete media was added. On day 7, media was replaced. BMDMs were harvested on day 10 using cell stripper solution for further experiments. Dendritic cells were isolated from the spleens of wildtype, using mouse CD11c UltraPure MicroBeads (130-125-835; Miltenyi Biotec) according to the manufacturer's instructions) and cultured in RPMI medium supplemented with 10% heat-inactivated charcoal-stripped FBS, 50 μM β-mercaptoethanol, 1% sodium pyruvate, 1% nonessential amino acids, % penicillin/streptomycin, and 1% Glutamax.

Cell Isolation. Fresh tumors, and lungs were collected separately from mice, and digested in DMEM/F12 supplemented with 2 mg/ml type II collagenase and 1% penicillin/streptomycin for 45 mins at 37° C. while shaking. Subsequently, cells were passed through a 70-μm filter into single-cell suspension, washed with FACS buffer, incubated with ACK lysis buffer for 1 min, and washed with FACS buffer before antibody staining for flow cytometry. Single cell suspensions of spleens were obtained by mechanical dissociation through a 70-μm filter in isolation buffer (DPBS supplemented with 0.5% BSA and 2 mM EDTA). Subsequently, cells were washed with isolation buffer, incubated with ACK lysis buffer, and washed with isolation buffer before antibody staining for flow cytometry or immune cell isolation. Cd11b+ (130-126-725) and Ly6G+ (130-120-337) were isolated from single cell suspensions using UltraPure MicroBeads from Miltenyi Biotec according to the manufacturer's instructions.

In vitro T_{reg} expansion assays. T cells were cultured in RPMI medium supplemented with 10% heat-inactivated charcoal-stripped FBS, 50 μM β-mercaptoethanol (Gibco), 1% sodium pyruvate, 1% nonessential amino acids, % penicillin/streptomycin, and 1% Glutamax (Gibco). Naïve CD4⁺ T cells were isolated from the spleens of wildtype and OT-II, using Naive CD4⁺ T Cell Isolation Kit (Miltenyi Biotec according to the manufacturer's instructions). T cells were labeled with the vital dye CFSE (BioLegend) or Cell Trace Violet (ThermoFisher) according to the manufacturer's instructions. For wildtype T_{reg} expansion in presence of antigen presenting cells, naïve CD4⁺ T cells were co-cultured with APC at indicated ratio in the presence of 0.5 μg/mL (anti-CD3 BioLegend), 0.5 ng/mL TGFβ (BioLegend) and 0.5 ng/ml IL-2 (BioLegend) for 72 hr at 37° C. For OT-II T_{reg} expansion, OTII naïve CD4⁺ T cells from OT-II mice were activated by antigen presentation by culturing BMDMs were primed with 10 μg/mL OVA323-339 (Bachem) and 0.5 μg/mL LPS (Sigma). naïve CD4+ T cells from OTII mice were co-cultured with primed APC at indicated ratio for 72 hrs. After 72 hrs of culture, cells were transferred into V-bottom plates (BD), pelleted by centrifugation and incubated with antibody staining mix containing Ghost Dye™ Red 780 viability dye diluted in PBS for 15 minutes at 4° C. Cell surface antigens were stained for 15 minutes at 4° C. with an antibody staining mix containing Ghost Dye™ Red 780 viability dye diluted in PBS. Cells were fixed and permeabilized with BD Cytofix/Cytoperm™ for 20 minutes at 4° C. Antibodies against intracellular antigens were diluted in 1×BD Perm/Wash™ buffer and cells were stained for 30 minutes at 4° C. Cytometry data were acquired on a LSRII, Fortessa, Symphony A1 (Becton Dickinson, NJ). Foxp3 induction was assessed by expression of FOXP3.

Gene Silencing and Overexpression. For gene knockdown small interfering RNA was delivered using HiPerFect transfection reagent (Qiagen). BMDMs were transfected with either negative control or Caspase-1/IL-1β-complementary siRNA (SMARTpool, ON-TARGETplus siRNA, Dharmacon) at 50 nM. Expression of the target genes after the knockdown was assessed using qPCR. For NR0B2 overexpression, NR0B2 overexpressing plasmid was delivered using Effectine transfection reagent (Qiagen). All transfections were conducted in accordance with the manufacturer's protocol. Media was changed 12 h post-transfection and cells were harvested for subsequent experiments 48 h post transfection.

Screening to identify NR0B2 agonists. HepG2 cells were co-transfected with ABCA1 renilla reporter plasmid and TK-control firefly luciferase vector at 20:1 using Lipofectamine 3000 reagent according to manufacturer's instructions for 24 hrs. Afterwards, 7000 cells were seeded in to 384 well f-bottom, white plates (Greiner Bio-One). Test compounds were added to the plates in a final working volume of 30 μL (for a final concentration of 50 μM) in presence of DMSO or 1 μM GW3965 and incubated for 20-24 hrs at 37° C. Luciferase activity was measured using the Luc-Pair Duo-Luciferase HT Assay kit (GeneCopoeia, Rockville, MD, USA) by adding luciferase substrates sequentially following manufacturer instructions. Luminescence was measured with 2 s integration times in a microplate reader (BioTek Cytation 5), 15 min after adding each substrate. The addition of reagents to the microplates was timed at each step to match the reading time delays and reading sequence of the microplate reader.

Resazurin cell viability assay. 20,000 cells were seeded into 96 well f-bottom, black plates (Corning Costar) and cell viability was measured using 100 μL resazurin per well at 0.3 mg/mL (Acros Organics). The fluorescence signal was quantified by plate reader (560 nm excitation/590 nm emission) at 2-3 h.

Inflammasome activation, RNA extraction, quantitative real PCR. When comparing inflammasome responses, BMDMs were treated with 500 ng/mL LPS for 4 h 37° C. in complete RPMI 1640. After 4 hr priming, 15 μM Nigericin was added for 45 mins at 37° C. Next, media was removed, washed with PBS and inflammasome activation was assessed using qPCR or inflammasome caspase-1 activity was measured using Caspase-Glo® 1 inflammasome assay kit (G9951; Promega) according to manufacturer's instructions.

Measurement of Intracellular Ca²⁺ Signaling. Intracellular Ca²⁺ was measured using a Fluo-4 direct calcium assay kit (F10471; Invitrogen) according to manufacturer's instructions. In brief, BMDMs, Cd11c⁺, or Cd11b⁺ cells were cultured in 96 well or 384 well black flat bottom plates (Corning Costar). The cells were incubated in 1× Fluo-4 Direct calcium reagent with probenecid for 30 mins at 37° C. and 5% CO₂ and 30 mins at room temperature. Then cells were stimulated with ionomycin or PMA at indicated concentrations, immediately measured the fluorescence signal for excitation at 494 nm and emission at 516 nm using BioTek Cytation 5 plate reader.

Measurement of Intracellular Cellular Oxidative Stress. Intracellular cellular oxidative stress was measured using CellROX™ Deep Red Reagent (C10422; Invitrogen) according to manufacturer's instructions.

Compound Uptake Assay. Cellular uptake of DSHN and DSHN-OMe was determined in RAW 264.7 cells cultured in 60×15 mm tissue culture dishes. Cells were treated with vehicle (0.1% DMSO), 50 μM DSHN or 50 μM DSHN-OMe for 4 h, 8 h, or 24 h. After incubation, cells were harvested, washed with PBS and cell pellets were incubated at −80° C. freezer. Next, cell pellets were resuspended in 200 μL 70:30 MeOH:H₂O and sonicated to lyse cells. Debris from lysed cell suspension was removed by centrifugation. Resulting supernatant was analyzed by LC-MS/MS.

Protein expression, purification, and thermal shift assay. A gene containing a hexa-His-TEV fusion with full length NR0B2 in pET21(a)+ was transformed in E. coli BL21(DE3). A single colony was used to inoculate a 100 mL starter culture of LB broth containing 100 μg/mL ampicillin, which was allowed to grow overnight with shaking at 37° C. This starter culture was used to inoculate flasks containing autoinducing media, which were grown at 37° C. with shaking until they reached an OD⁶⁰⁰ of 0.8. Subsequently, the temperature was reduced to 16° C. and cells were allowed to grow for another 24 hours. After harvesting by centrifugation, cells were resuspended at 15 mL/g cell paste in a buffer comprised of 25 mM HEPES pH 8.0, 250 mM NaCl, 20 mM imidazole pH 8.0, 5% glycerol, 0.5 mM TCEP, and Roche EDTA-free protease inhibitor cocktail. Cells were lysed by sonication then centrifuged at 18,000×g for 30 minutes to remove insoluble material. The lysate was loaded onto a gravity flow Ni-NTA column, washed with 5 column volumes (CVs) of resuspension buffer, and protein was eluted using the resuspension buffer supplemented with 500 mM imidazole. Collected protein was dialyzed overnight in the resuspension buffer without imidazole then concentrated and purified on a Superdex 200 HiLoad 200 16/600 size exclusion column. A peak corresponding to ~29 kDa was collected and molecular weight was verified using SDS-PAGE. For thermal shift assays, vehicle (DMSO), 1 mM DSHN-OMe, or 1 mM DSHN was incubated with 6 μM purified NR0B2 overnight at 4° C. We found that this concentration was the minimal amount required to give a reliable melt curve. The next morning the mixtures were centrifuged at 20,000×g for 30 minutes to remove any insoluble protein or ligand. The supernatant was mixed with SYPRO orange (Thermo) then placed in 96-well qPCR plates in triplicate. Melt curves were obtained with an Applied Biosystems qPCR machine between 25 and 95° C. at a gradient of 0.15° C./second. Melt curve data were fit using Thermo Protein Thermal Shift Software. These experiments were performed three independent times with three technical replicates each.

Human Tumor Specimens. Deidentified serial breast cancer sections and corresponding subtype information were obtained from archival collections at the Département de Biologie et de Pathologie des Tumeurs, Centre Georges-François Leclerc, Dijon, France. They were stained and analyzed by the Research Histology and Tissue Imaging Core, University of Illinois at Chicago, Illinois, USA. Three consecutive sections from each sample were stained with dual immunostain for FoxP3 and panCK, RNAscope assay with NR0B2 probe, and dual immunostain for CD8 and panCK. Staining for all targets was performed on BOND RX autostainer (Leica Biosystems, Deer Park, IL) using preset protocols. For dual immunostaining, the first and third slides were stained with either FoxP3 (1:100, #12653, Cell Signaling Technology, Danvers, MA) or CD8 (1:100, #ACI3160, Biocare Medical, Pacheco, CA) antibodies using BOND Polymer Refine Detection kit (#DS9800), followed by staining with panCK antibody (1:4000, #M351501, Agilent, Santa Clara, CA) using BOND Polymer Red Detection Kit (#DS9390). The middle slide was stained with RNAscope 2.5 LS Probe—Hs-NR0B2 assay (#877508, Advanced Cell Diagnostics, Newark, CA) using RNAscope 2.5 Leica assay-RED reagents for hybridization and detection. The standard ACD Red Rev BOND Rx protocol was modified to include BOND Epitope Retrieval Solution 2 (Leica Biosystems) pretreatment for 25 min at 95° C. and to increase Amp5 step time to 30 minutes. Positive (Hs-PPIB, #313908) and negative (DapB, #3120) probe assays were included with each batch of samples. The stained slides were scanned at 40× magnification on PhenolMager HT (Akoya Biosciences, Malborough, MA). All three slides were registered using HALO software (Indicalabs, Albuquerque, NM). HALO algorithms were used to segment tumor from stroma and count positive T cells and NR0B2 transcripts, at single-cell resolution. We computed Spearman correlation coefficients to evaluate associations between CD8 and FoxP3 density and NR0B2 expression in the tumor and stromal compartments. These correlations were also computed within strata defined by ER/PR, HER2 and triple negative status. CD8, FoxP3 and NR0B2 expression was compared among the three subtypes using pairwise two-sided multiple comparison-adjusted analysis. All P values were two-sided with a 0.05 threshold for statistical significance.

### Example 2. Preparation and Characterization of DSHN Derivatives (Corresponding Derivative Numbers are Outlined in Table 4)

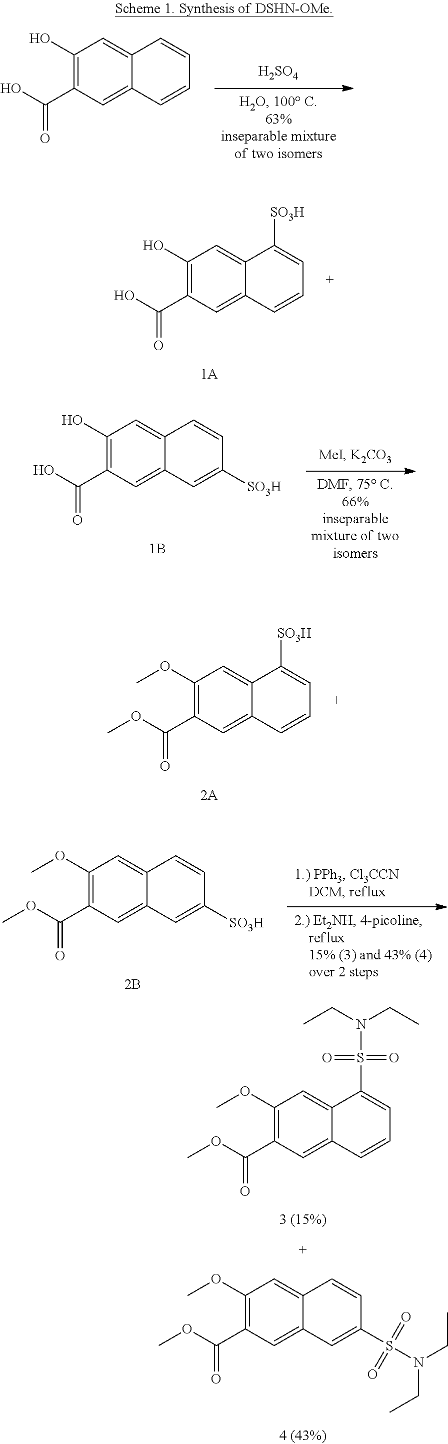

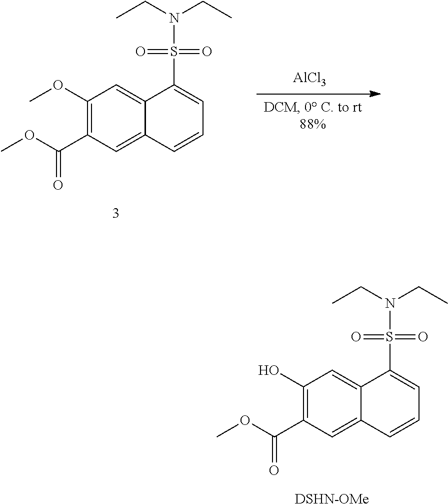

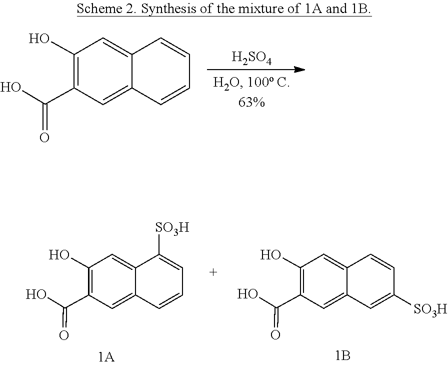

3-Hydroxy-5-sulfo-2-naphthoic acid (1A) and 3-hydroxy-7-sulfo-2-naphthoic acid (1B). Procedure: To a 50 mL round bottom flask was added concentrated sulfuric acid (15 mL, 280 mmol, 5.3 eq.) and water (10 mL). The solution was heated to 100° C. and 3-hydroxy-2-naphthoic acid (10 g, 52.88 mmol, 1.0 eq.) was added. The reaction was stirred at 100° C. for 3 hours. The reaction mixture was then removed from heat and concentrated in vacuo to remove water. The crude product was purified via reverse phase flash column chromatography (H₂O to 1:1 H₂O/Acetonitrile) yielding a 1:3 mixture of isomers 1A and 1B, respectively, as a yellow solid (9 g, 63%). ¹H NMR (500 MHz, Methanol-d4) δ Major (1B): 8.62 (s, 3H), 8.32 (s, 3H), 7.89 (d, J=8.7 Hz, 3H), 7.77 (d, J=8.7 Hz, 3H), 7.30 (s, 3H). Minor (1A): 8.58 (s, 1H), 8.24 (s, 1H), 8.18 (d, J=7.2 Hz, 1H), 7.96 (d, J=8.2 Hz, 1H), 7.34 (t, J=7.7 Hz, 1H). ¹³C NMR (125 MHz, Methanol-d4) δ Major (1B): 172.91, 159.39, 139.72, 134.80, 127.90, 127.66, 127.17, 127.08, 123.24, 116.81, 112.26. Minor (1A): 172.84, 158.51, 134.75, 134.50, 134.12, 133.79, 129.78, 129.23, 116.10, 112.51, 112.02. HRMS (ESI): calc, for C₁₁H₇O₆S [M−H]⁻: 266.9964, found: 266.9959.

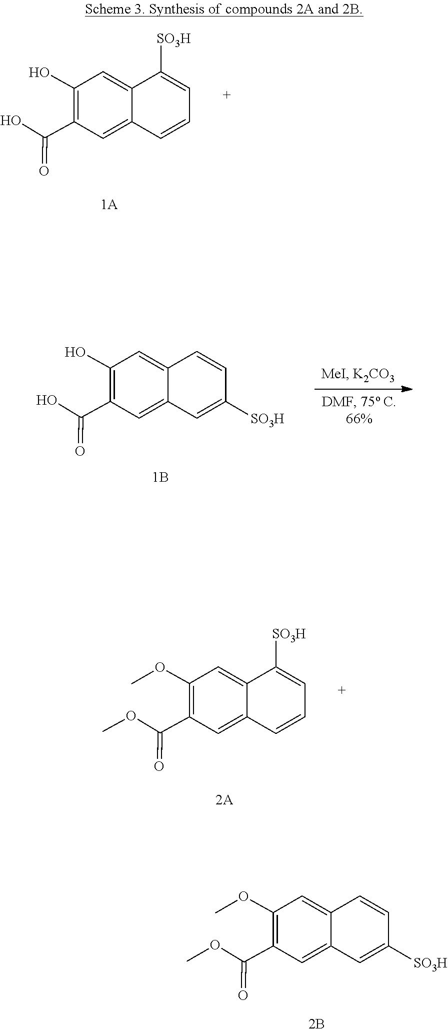

7-Methoxy-6-(methoxycarbonyl) naphthalene-1-sulfonic acid (2A) and 6-methoxy-7-(methoxycarbonyl) naphthalene-2-sulfonic acid (2B). Procedure: To an oven dried 200 mL round bottom flask was added a mixture of isomers 1A and 1B (1.1 g. 4.1 mmol, 1.0 eq.) and K₂CO₃ (4 g, 28.7 mmol, 7.0 eq.). The flask was purged with nitrogen and DMF (18 mL, 0.25 M) was added. Methyl iodide (2.3 mL, 36.9 mmol, 9.0 eq.) was added dropwise and the reaction was subsequently heated to 75° C. and stirred for 2 hours. Upon completion, the reaction was cooled to room temperature and quenched with 50 mL of methanol. The crude mixture was azeotroped via rotary evaporation with toluene (3×50 mL) to remove DMF. The crude product was purified via reverse phase flash column chromatography (H₂O to acetonitrile) to yield a 1:3.5 mixture of isomers 2A and 2B, respectively, as a yellow solid (2.9 g, 66%). ¹H NMR (500 MHz, Methanol-d4) δ Major (2B): 8.29 (s, 3.5H), 8.27 (s, 3.5H), 7.89 (dd, J=8.7, 1.8 Hz, 3.5H), 7.84 (d, J=8.6 Hz, 3.5H), 7.38 (s, 3.5H), 3.93 (s, 10.5H), 3.89 (s, 10.5H). Minor (2A): 8.31 (s, 1H), 8.27 (s, 1H), 8.18 (dd, J=7.3, 1.3 Hz, 1H), 7.93 (d, J=8.2 Hz, 1H), 7.37-7.34 (m, 1H), 4.00 (s, 3H), 3.93 (s, 3H). ¹³C NMR (125 MHz, Methanol-d4) δ Major (2B): 166.79, 156.64, 136.86, 132.55, 126.80, 126.25, 125.85, 125.24, 122.79, 122.65, 106.59, 55.08, 51.45. Minor (2A): 166.82, 155.82, 140.64, 139.18, 132.18, 131.80, 131.72, 128.29, 127.76, 121.88, 106.06, 55.01, 51.41. HRMS (ESI): calc, for C₁₃H₁₃O₆S [M+H]⁺: 297.0432, found: 297.0421.

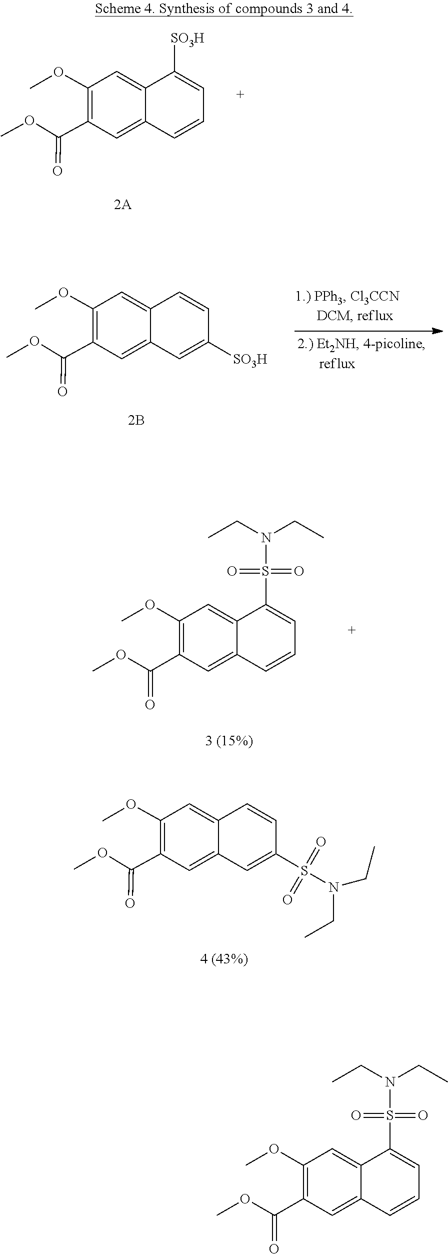

Methyl 5-(N,N-diethylsulfamoyl)-3-methoxy-2-naphthoate (3). Procedure: In an oven dried three neck 25 mL round bottom flask, a mixture of 2A and 2B (250 mg, 0.75 mmol, 1.0 eq.) was dissolved in DCM (2.5 mL). A reflux condenser was attached, and the entire apparatus was purged with nitrogen. Cl₃CCN (250 μL, 2.53 mmol, 3.0 eq.) was added dropwise at room temperature. The reaction was then heated to 45° C. and stirred for 10 minutes before addition of PPh₃ (660 mg, 2.53 mmol, 3.0 eq.) in 2.5 mL of DCM. The reaction was then stirred at reflux for an additional 5 hours until consumption of starting material was observed by TLC. Et₂NH (262 μL, 2.53 mmol, 3.0 eq.) and freshly distilled 4-picoline (760 μL, 7.6 mmol, 9.0 eq.) were added dropwise as a mixture to the reaction at reflux. After 2 hours, the reaction was diluted with DCM (100 mL) and washed with 1 M HCl (50 mL, 5×). The organic layer was washed with saturated sodium bicarbonate and brine, then dried over sodium sulfate, and concentrated in vacuo. The crude product was purified by column chromatography (hexanes to 3:1 hexanes/ethyl acetate) to yield the isolated products 3 (48 mg, 15%) as a light brown solid and 4 (137 mg, 43%) as a light brown solid. ¹H NMR (500 MHz, CDCl₃) δ 8.30 (s, 1H), 8.19 (dd, J=7.4, 1.3 Hz, 1H), 8.05 (s, 1H), 7.97 (d, J=8.1 Hz, 1H), 7.38 (t, J=7.8 Hz, 1H), 4.01 (s, 3H), 3.93 (s, 3H), 3.33 (q, J=7.2 Hz, 4H), 1.04 (t, J=7.1 Hz, 6H). ¹³C NMR (125 MHz, CDCl₃) δ 166.09, 156.86, 134.49, 133.79, 133.23, 131.77, 131.64, 128.55, 122.71, 122.61, 104.85, 56.08, 52.47, 40.98, 13.78. HRMS (ESI): calc, for C₁₇H₂₂NO₅S [M+H]⁺: 352.1218, found: 352.1227.

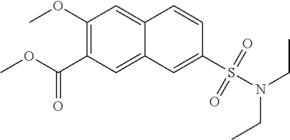 [SMILES: CCN(CC)S(=O)(=O)c1ccc2cc(OC)c(C(=O)OC)cc2c1]

Methyl 7-(N,N-diethylsulfamoyl)-3-methoxy-2-naphthoate (4). ¹H NMR (500 MHz, Methanol-d4) δ 8.42 (s, 1H), 8.41 (d, J=1.8 Hz, 1H), 8.00 (d, J=8.7 Hz, 1H), 7.85 (dd, J=8.7, 1.9 Hz, 1H), 7.51 (s, 1H), 4.02 (s, 3H), 3.95 (s, 3H), 3.32 (m, 4H), 1.14 (t, J=7.1 Hz, 6H). 13C NMR (125 MHz, Methanol-d4) δ 168.00, 158.90, 139.13, 137.24, 134.20, 129.87, 129.15, 127.63, 126.05, 125.13, 108.00, 56.59, 52.94, 43.42, 14.69. HRMS (ESI): calc, for C₁₇H₂₂NO₅S [M+H]⁺: 352.1218, found: 352.1216.

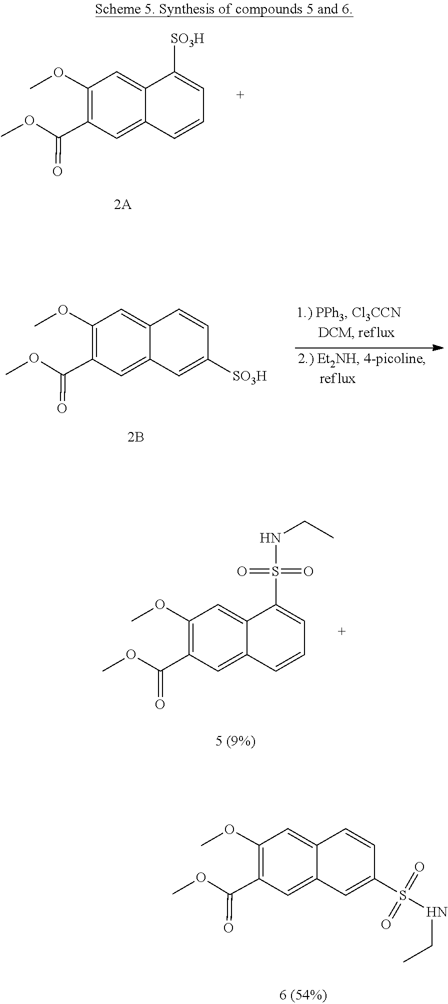 [SMILES: COC(=O)c1cc2cccc(S(=O)(=O)O)c2cc1OC ; COC(=O)c1cc2cc(S(=O)(=O)O)ccc2cc1OC ; CCNS(=O)(=O)c1cccc2cc(C(=O)OC)c(OC)cc12 ; CCNS(=O)(=O)c1ccc2cc(OC)c(C(=O)OC)cc2c1]

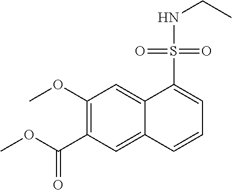 [SMILES: CCNS(=O)(=O)c1cccc2cc(C(=O)OC)c(OC)cc12]

Methyl 5-(N-ethylsulfamoyl)-3-methoxy-2-naphthoate (5). Procedure: In an oven dried, three neck, 25 mL round bottom flask, a mixture of 2A and 2B (330 mg, 0.99 mmol, 1.0 eq.) was dissolved in DCM (3 mL). A reflux condenser was attached, and the entire apparatus was purged with nitrogen. Cl₃CCN (330 μL, 2.97 mmol, 3.0 eq.) was added dropwise at room temperature. The reaction was then heated to 45° C. and stirred for 10 minutes before addition of PPh₃ (870 mg, 3.34 mmol, 3.0 eq.) in 3 mL of DCM. The reaction was then stirred at reflux for an additional 5 hours until consumption of starting material observed by TLC. EtNH₂ (346 μL, 2.97 mmol, 3.0 eq.) and freshly distilled 4-picoline (1 mL, 10.0 mmol, 9.0 eq.) were added dropwise as a mixture to the reaction at reflux. After 2 hours, the reaction was diluted with DCM (100 mL) and washed with 1 M HCl (50 mL, 5×). The organic layer was washed with saturated sodium bicarbonate and brine, then dried over sodium sulfate, and concentrated in vacuo. The crude product was purified by column chromatography (hexanes to 3:1 hexanes/ethyl acetate) to yield the isolated products 5 (29 mg, 9%) as a white solid and 6 (173 mg, 54%) as a white solid. ¹H NMR (500 MHz, DMSO-d₆) δ 8.42 (s, 1H), 8.26 (d, J=8.1 Hz, 1H), 8.18 (dd, J=7.3, 1.3 Hz, 1H), 8.06 (s, 1H), 7.96 (t, J=5.7 Hz, 1H), 7.55 (t, J=7.8 Hz, 1H), 3.99 (s, 3H), 3.87 (s, 3H), 2.80 (qd, J=7.2, 5.5 Hz, 2H), 0.90 (t, J=7.2 Hz, 3H). ¹³C NMR (125 MHz, DMSO-d₆) δ 165.70, 155.77, 134.27, 134.01, 132.20, 130.84, 130.31, 128.08, 123.06, 122.93, 104.38, 55.99, 52.36, 37.33, 15.00. HRMS (ESI) calc, for C₁₅H₁₈NO₅S [M+H]⁺: 324.0905, found: 324.0908.

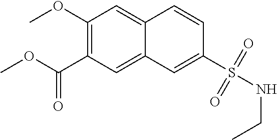 [SMILES: CCNS(=O)(=O)c1ccc2cc(OC)c(C(=O)OC)cc2c1]

Methyl 7-(N-ethylsulfamoyl)-3-methoxy-2-naphthoate (6). ¹H NMR (500 MHz, CDCl₃) δ 8.37 (d, J=1.8 Hz, 1H), 8.33 (s, 1H), 7.87 (dd, J=8.7, 1.8 Hz, 1H), 7.81 (d, J=8.7 Hz, 1H), 7.23 (s, 1H), 4.01 (s, 3H), 3.95 (s, 3H), 3.02 (m, J=6.9 Hz, 2H), 1.09 (t, J=7.2 Hz, 3H). (N—H proton exchanges and is therefore not visible on 1H spectrum). 13C NMR (125 MHz, CDCl₃) δ 166.12, 157.86, 137.75, 135.52, 133.68, 129.15, 127.98, 126.16, 124.99, 123.65, 106.84, 56.26, 52.62, 38.39, 15.17. HRMS (ESI): calc, for C₁₅H₁₈NO₅S [M+H]⁺: 324.0905, found: 324.0908.

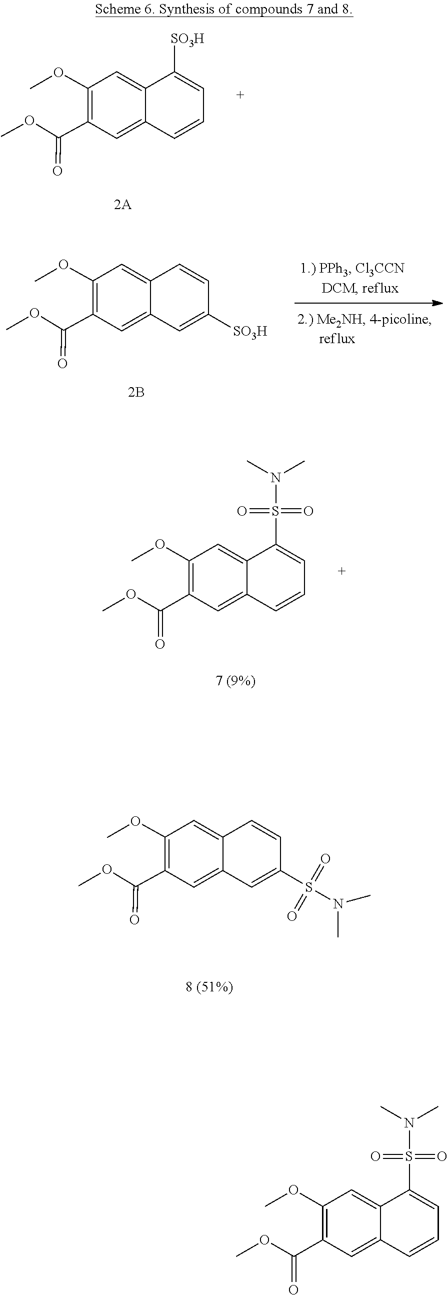

Methyl 5-(N,N-dimethylsulfamoyl)-3-methoxy-2-naphthoate (7). Procedure: In an oven dried, three neck, 25 mL round bottom flask, a mixture of 2A and 2B (330 mg, 0.99 mmol, 1.0 eq.) was dissolved in DCM (3 mL). A reflux condenser was attached, and the entire apparatus was purged with nitrogen. Cl₃CCN (330 μL, 2.97 mmol, 3.0 eq.) was added dropwise at room temperature. The reaction was then heated to 45° C. and stirred for 10 minutes before addition of PPh₃ (870 mg, 3.34 mmol, 3.0 eq.) in 3 mL of DCM. The reaction was then stirred at reflux for an additional 5 hours until consumption of starting material was observed by TLC. Me₂NH (356 μL, 2.97 mmol, 3.0 eq.) and freshly distilled 4-picoline (1 mL, 10.0 mmol, 9.0 eq.) were added dropwise as a mixture to the reaction at reflux. After 2 hours, the reaction was diluted with DCM (100 mL) and washed with 1 M HCl (50 mL, 5×). The organic layer was washed with saturated sodium bicarbonate and brine, then dried over sodium sulfate, and concentrated in vacuo. The crude product was purified by column chromatography (hexanes to 3:1 hexanes/ethyl acetate) to yield the isolated products 7 (28 mg, 9%) as a white solid and 8 (166 mg, 51%) as a white solid. ¹H NMR (500 MHz, CDCl₃) § 8.34 (s, 1H), 8.25 (s, 1H), 8.22 (d, J=7.4 Hz, 1H), 8.04 (d, J=8.1 Hz, 1H), 7.46 (t, J=7.7 Hz, 1H), 4.04 (s, 3H), 3.97 (s, 3H), 2.82 (s, 6H). ¹³C NMR (125 MHz, CDCl₃) δ 166.23, 157.10, 134.95, 133.29, 132.71, 132.28, 131.18, 128.71, 122.87, 122.81, 105.22, 56.22, 52.65, 37.64. HRMS (ESI): calc, for C₁₅H₁₈NO₅S [M+H]⁺: 324.0905, found: 324.0907.

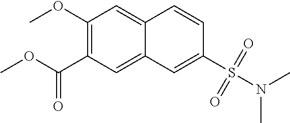

Methyl 7-(N,N-dimethylsulfamoyl)-3-methoxy-2-naphthoate (8). ¹H NMR (500 MHz, CDCl₃) δ 8.36 (s, 1H), 8.27 (s, 1H), 7.84 (d, J=8.7 Hz, 1H), 7.77 (dd, J=8.6, 1.8 Hz, 1H), 7.25 (s, 1H), 4.01 (s, 3H), 3.94 (s, 3H), 2.72 (s, 6H). ¹³C NMR (125 MHz, CDCl₃) δ 166.02, 157.83, 137.76, 133.66, 131.30, 129.71, 127.66, 126.22, 125.57, 123.73, 106.77, 56.25, 52.57, 38.03. HRMS (ESI): calc, for C₁₅H₁₈NO₅S [M+H]⁺: 324.0905, found: 324.0898.

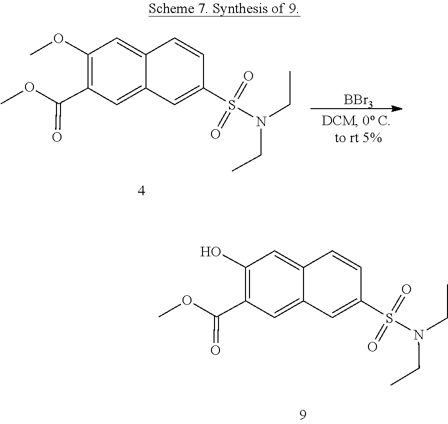

Methyl 7-(N,N-diethylsulfamoyl)-3-hydroxy-2-naphthoate (9). Procedure: To an oven dried 4 mL scintillation vial was added 4 (11.5 mg, 0.03 mmol, 1.0 eq.). The flask was purged with nitrogen, DCM (0.2 mL, 0.15 M) was added, and the reaction was cooled to 0° C. BBr₃ (0.14 mL, 0.18 mmol, 3.3 eq.) was added dropwise as a 1 M solution in DCM. The ice bath was removed, and the reaction was stirred at room temperature for 16 hours. The reaction mixture was then poured into ice water (15 mL) and extracted with ethyl acetate (15 mL, 3×). The combined ethyl acetate layers were then washed with brine, dried over sodium sulfate, and concentrated in vacuo. The crude product was purified with reverse phase flash column chromatography (H₂O to acetonitrile) to yield 9 as a light brown solid (0.53 mg, 5%). ¹H NMR (500 MHz, CDCl₃) δ 10.68 (s, 1H), 8.61 (s, 1H), 8.35 (d, J=1.2 Hz, 1H), 7.77 (s, 1H), 7.77 (s, 1H), 7.36 (s, 1H), 4.06 (s, 3H), 3.29 (q, J=7.2 Hz, 4H), 1.14 (t, J=7.1 Hz, 6H). ¹³C NMR (125 MHz, CDCl₃) δ 169.97, 158.70, 139.18, 135.74, 133.96, 129.86, 127.81, 125.71, 125.66, 115.76, 112.25, 53.09, 42.13, 14.34. HRMS (ESI): calc, for C₁₆H₂₀NO₅S [M+H]⁺: 338.1062, found: 338.1061.

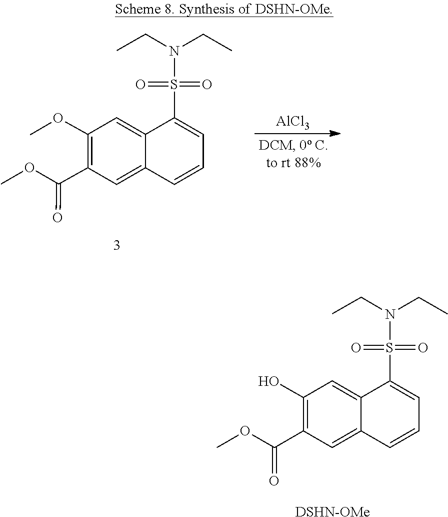

Methyl 5-(N,N-diethylsulfamoyl)-3-hydroxy-2-naphthoate (DSHN-OMe). Procedure: To an oven dried 4 mL scintillation vial was added 3 (50 mg, 0.142 mmol, 1.0 eq.). The vial was purged with nitrogen and DCM (1 mL, 0.2 M) was added. The reaction was cooled to 0° C. and AlCl₃ (47 mg, 0.355 mmol, 2.5 eq) was added in one portion. The ice bath was removed, and the reaction was stirred at room temperature for 2.5 hours until full consumption of starting material was observed by TLC. The reaction mixture was diluted with H₂O and DCM (20 mL each). The organic layer was washed with saturated sodium bicarbonate and brine, then dried over sodium sulfate and concentrated in vacuo. The crude material was then recrystallized from minimal DCM and hexanes to afford DSHN-OMe as a light brown solid (43 mg, 88%). ¹H NMR (500 MHz, CDCl₃) δ 10.55 (s, 1H), 8.54 (s, 1H), 8.28 (dd, J=7.3, 1.3 Hz, 1H), 8.06 (s, 1H), 7.99 (d, J=8.2 Hz, 1H), 7.37 (dd, J=8.2, 7.3 Hz, 1H), 4.05 (s, 3H), 3.38 (q, J=7.1 Hz, 4H), 1.08 (t, J=7.1 Hz, 6H). ¹³C NMR (125 MHz, CDCl₃) & 169.84, 157.98, 135.20, 133.99, 133.30, 133.28, 133.26, 128.11, 122.25, 115.01, 110.49, 53.00, 40.79, 13.72. HRMS (ESI): calc, for C₁₆H₂₀NO₅S [M+H]⁺: 338.1062, found: 338.1059.

### Example 3. Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | mg/tablet |
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |
| iii) Capsule | mg/capsule |
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |
| (iv) Injection 1 (1 mg/mL) | mg/mL |
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |
| (v) Injection 2 (10 mg/mL) | mg/mL |
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Additionally, the specific ingredients and proportions are for illustrative purposes. Ingredients may be exchanged for suitable equivalents and proportions may be varied, according to the desired properties of the dosage form of interest.

Additional details and useful techniques are described in PCT publication WO 2020/198576 (Nelson), PCT publication WO 2018/177151 (Li), and U.S. Pat. No. 9,682,939 (Chianelli), which are incorporated herein by reference.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A compound of formula I: 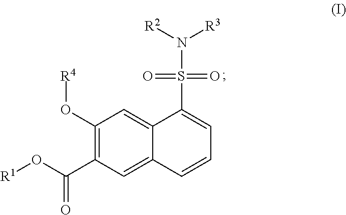
wherein
R¹ is (C₁-C₈)alkyl or H;
R² is ethyl, (C₁-C₈)alkyl, or H;
R³ is ethyl, (C₁-C₈)alkyl, or H; or
R² and R³ taken together with the nitrogen atom to which they are attached form a 3- to 9-membered ring heterocycle provided that R¹ is (C₁-C₈)alkyl; and
R⁴ is H or (C₁-C₈)alkyl;
wherein the naphthalene moiety of formula I and each (C₁-C₈)alkyl are optionally substituted with one or more substituents;
or a pharmaceutically acceptable salt thereof;
provided that R¹ is (C₁-C₈)alkyl when R² and R³ are both ethyl and R⁴ is H; and
provided that R¹, R², R³, and R⁴ are not all H.

2. The compound of claim 1, wherein R¹ is (C₁-C₃)alkyl.

3. The compound of claim 1, wherein R² is (C₁-C₃)alkyl and R³ is (C₁-C₃)alkyl.

4. The compound of claim 1, wherein R¹ is methyl or ethyl.

5. The compound of claim 1, wherein R² and R³ are each ethyl, methyl, or propyl.

6. The compound of claim 1, wherein R¹ is methyl and R² and R³ are each ethyl or methyl.

7. The compound of claim 1, wherein the compound of formula I is: 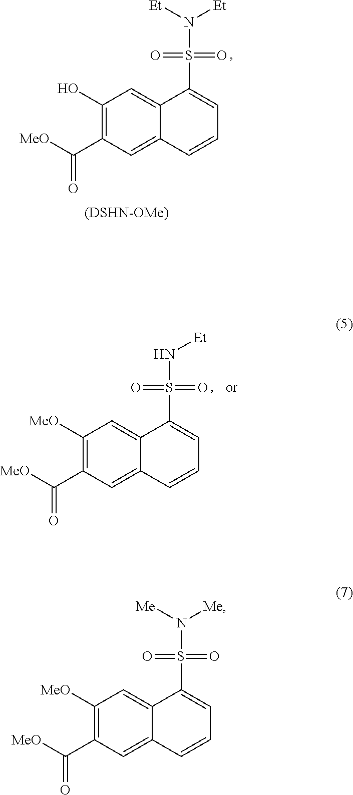
or a pharmaceutically acceptable salt thereof.

8. A compound of formula II: 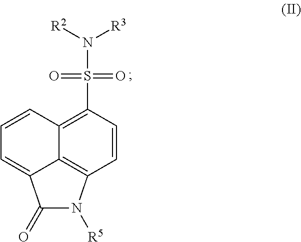
wherein
R² is (C₁-C₈)alkyl;
R³ is (C₁-C₈)alkyl; or
R² and R³ taken together with the nitrogen atom to which they are attached form a 3- to 9-membered ring heterocycle; and
R⁵ is H or (C₁-C₈)alkyl;
wherein the naphthalene moiety of formula II and each (C₁-C₈)alkyl are optionally substituted with one or more substituents;
or a pharmaceutically acceptable salt thereof; or
a compound of formula III: 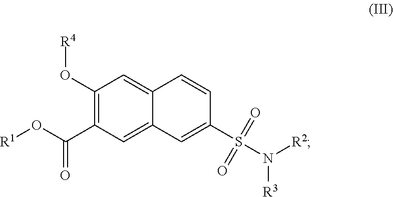
wherein
R¹ is (C₁-C₈)alkyl or H;
R² is (C₁-C₈)alkyl or H;
R³ is (C₁-C₈)alkyl or H; or
R² and R³ taken together with the nitrogen atom to which they are attached form a 3- to 9-membered ring heterocycle; and
R⁴ is (C₁-C₈)alkyl or H;
wherein the naphthalene moiety of formula I and each (C₁-C₈)alkyl are optionally substituted with one or more substituents;
or a pharmaceutically acceptable salt thereof.

9. The compound of claim 8, wherein the compound of formula II is: 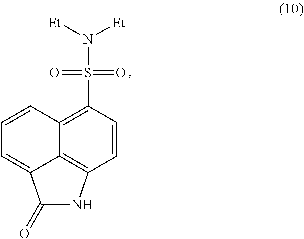 [SMILES: CCN(CC)S(=O)(=O)c1ccc2c3c(cccc13)C(=O)N2]
or a pharmaceutically acceptable salt thereof; or
wherein the compound of formula III is: 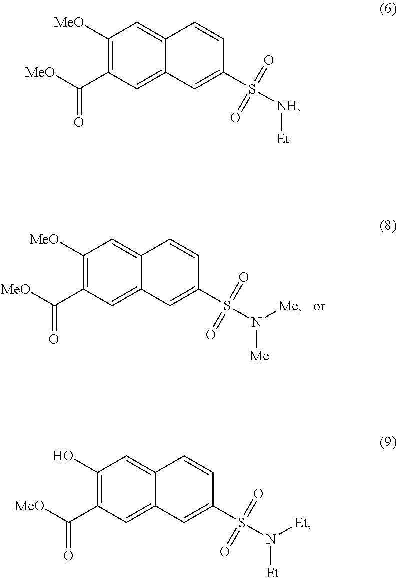 [SMILES: CCNS(=O)(=O)c1ccc2cc(OC)c(C(=O)OC)cc2c1 ; COC(=O)c1cc2cc(S(=O)(=O)N(C)C)ccc2cc1OC ; "or" ; CCN(CC)S(=O)(=O)c1ccc2cc(O)c(C(=O)OC)cc2c1]
or a pharmaceutically acceptable salt thereof.

10. A pharmaceutical composition comprising the compound of claim 1 and a pharmaceutically acceptable diluent or carrier.

11. A method of treating cancer comprising administering to a subject in need thereof a therapeutically effective amount of a compound of claim 1, wherein the cancer is thereby treated.

12. The method of claim 11, wherein administering the compound results in inhibition of T_{reg} expansion.

13. The method of claim 11, wherein the administering results in depletion of regulatory T cells, depletion of suppressor T cell, depletion of immune suppressive T cells, depletion of scurfin positive T cells, depletion of FoxP3+ cells (forkhead box 3 positive cells), or a combination thereof.

14. The method of claim 11, wherein the administering results in an expansion of cytotoxic T cells.

15. The method of claim 11, wherein the administering results in agonism of a regulatory nuclear receptor wherein the regulatory nuclear receptor is NR0B2 (nuclear receptor subfamily 0, group B, member 2).

16. The method of claim 11, wherein the cancer is breast cancer, pancreatic cancer, lung cancer, melanoma, skin cancer, hematopoietic cancer, leukemia, lymphoma, colon cancer, rectal cancer, kidney cancer, renal cancer, urinary bladder cancer, oral cavity cancer, pharynx cancer, thyroid cancer, head and neck cancer, brain cancer, bone cancer, splenic cancer bladder cancer, esophageal adenocarcinoma, glioblastoma, hepatocellular carcinoma, urothelial cancer, muscle cancer, sarcoma, rhabdomyosarcoma, ovarian cancer, cervical cancer, uterine cancer, or prostate cancer.

17. The method of claim 11, wherein the compound activates a transcription factor.

18. The method of claim 17, wherein the transcription factor is farnesoid X receptor (FXR), estrogen receptor alpha (ERα), estrogen receptor beta (ERβ), liver x receptor alpha (LXRα), liver x receptor beta (LXRβ), liver receptor homolog 1 (LRH-1), or nuclear factor-κB (NF-κB).

19-22. (canceled)

23. A method of inhibiting the progression of a cancer comprising, contacting regulatory nuclear receptor NR0B2 (nuclear receptor subfamily 0, group B, member 2) and an effective amount of a compound that is an NR0B2 agonist, wherein the regulatory nuclear receptor that is agonized by the agonist is present within the microenvironment of the cancer and progression of the cancer is thereby inhibited.

24. The method of claim 23, wherein the NR0B2 agonist is a compound of formula I: 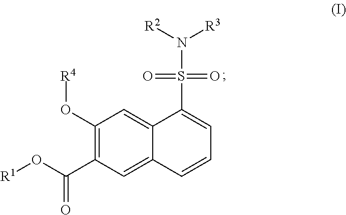
wherein
R¹ is (C₁-C₈)alkyl or H;
R² is ethyl, (C₁-C₈)alkyl, or H;
R³ is ethyl, (C₁-C₈)alkyl, or H; or
R² and R³ taken together with the nitrogen atom to which they are attached form a 3- to 9-membered ring heterocycle provided that R¹ is (C₁-C₈)alkyl; and
R⁴ is H or (C₁-C₈)alkyl;
wherein the naphthalene moiety of formula I and each (C₁-C₈)alkyl are optionally substituted with one or more substituents;
or a pharmaceutically acceptable salt thereof;
provided that R¹ is (C₁-C₈)alkyl when R² and R³ are both ethyl and R⁴ is H; and
provided that R¹, R², R³, and R⁴ are not all H.

25-27. (canceled)
